SEQUENCE LISTING (50 sequences)

SEQ ID NO: 1 - DNA, 3081 bp, C. elegans
  CDS (1)..(3078)
atgtgcgtgtcctccgctgtctgcgtgctctctcgtctctctctcgaacgtttttcaaatttttccgcgaattttgtggataattttcttcttgtggctcgttttgcacgtggtgaaaccttgaaaaacgggattgtgttggaaatttcaaaaatttgcccaaaactgtacatttctcctgtacccaaactctttgactgctccacagacaatcttctatttatttatattttatttgcatccaaagtgcgaatgaaagaaatgtcaacaccaacgggtcttcctgcgctaaatggcgatgtgttgagcgccatcaatgatatgatcggacgtgtaattatcattaatactacggacaaaaagcgatattctggggtgctgggcgccgtgtctcaggatttcgatttcgggatgcagtgcgtcgtggaaattacgaaggagaacgagaataacttgctgagaacagagtcggaatgcagagacaagatggtatttcactatagtgatattgtcgattttgcgtacgttacgcaagagatcaagaagcagcatgctgtcagcaaattcgtcaccgatagacaataccacggagatactccaatcgagggcgaagagcttcaggaatggaacggaggtgaggaggacggactcggaggatccattgaggacgacgtcgtcgttgctggtggacaaaccgccgctcgtcgcagcaacaatcacaacaacggcaccggatggtcggtcaacgacatgttcgccgccaacgagaagatgaacgtggtgtcgacgttcaaagaggatttaacgcaatacacaaccgtcgaagtggtaggaaccgacgaagatcgagcacgcgccgagcggctcgctagagaaattgagtccaactcgtcgtcgaagtttatggcgaacttggagaacgacgacgacgagcgagatttggacaaaatcacgcgtcaagaggatttcgagaatggcaatggccgaaagcgcaataataacagcttcaaccagcaacaacaacagcgccgcaatccgaatattgcaccaaacgggcagccagtgaatcgacgtgccgagggtcttcgcggagatcgtcgtaatagtggaagctcttcagcgaacaatagtcgctacggtgctccagcagcggctcagcagaactattcacagaatcagcagcagcagcaaggtcaaaaaggttatcgccgccagaacgaggaaaatgactggcagatggccaaggggaagggccagaaccagggccacgaccactcgttccgtcaacagcagaagcagatgctggatccgagacccaacaacaatgtgaagccggctgacgataaagctcaatctgccaccactgccactgctgctgccggtggatcccgtgtgacagatctcaagaactggggcaacgagttttcgattgcgacggctccaaaggatcaggctccggcggtgccagctggaaactcgggtagtgcctggaatcgcggaccaccgagctcgctggttgccaagggatcatcaaatgagtcgactccaccaccgacaaccaatggagaggaggctgagactaaaaaagaagaagctccatccacaagcgtcgatgtggcagctgcaccagtgcaaaacgtacagaacgacgcggaaaagcatcaggaagacgacaatgtttccgttacttcagagaatgattcagtgatcacttcgaagtcatcgtcgttcaaattcaacatcaatgctccagagttcaagccacgtgtggctccggcgactccaacagcgacgactccagtgcaaaatgaatatcatccacaacaacagccacatccagcaatgatggctccacaacaaggaccacctgctcctggaatgggaatggttccaccacacatgggcggcccacaaaatcaaggtcaaccaccgatgatgatgtggcaacagactggacaacagcaacaaggcggtggcggctatccacagaatcatcaattcccaattcaacatgtcccaatgcaaggtgttcctggacagatgtatggcccgggagctgcgactccggtcactgttgctcagcagccaaatcagcagcatcaggtgccaaccagtgcggctggtggtcagaatcatcagttgagagatggagagtatcgtgagaagcagccactctacatgccatatggaccgccacaaatggttccagtcacatctcagcaattctatcattcacagtaccaaggccagatgcagcaagctgctccataccaaatgaagatgatgccacagcaagctccacagggtgcctatcaacagagatatcagcagccacaagtgtacatgatgcctccacagggtcagcagcagcagccgagataccagggtcctccaccaccacagcaacagcaacagcaacaaccgcagcaacaacaattctccggagagcaaagtcgcccacaatcacatccgaatagtcagccaacgactccgggaccacgtggcgagttgccaaagatgagcggtgctccacagaacggaaacatgcaggccgagtcgagcagcaacgctagtcacagcggatcgacgagctctcagtcgggacaacgaagcggatctccaccaggtgctgttcctccaccaccaccaccacagcagcagcatcagcaacaacaacatccaccacatcatgctcctccacacgtcggagcaccaccaccacagatgatgcagcagcagcagcaacacattcaacagtatatgcaagtattctatccgatgataatgccccaacagatgccaatgcaacagaaccaacatcctcaacaaagtcttatgggcgagcgatctgatcaaggattcccgacgtcgggatactttgactaccgtacaatgccaaactatcagcaacaacaacaacagcaacagcaacaaatgcaccgccagaattcgcttccacagcagttccaaggcaatcaaggtgtaaatccatctggtcaacaatctggtccaccaccaccgccaccaccgtctcaacaaggcacaccacgtgatcagcagcactctcagagccccccgtaa

SEQ ID NO: 2 - protein, 1026 aa, C. elegans
MCVSSAVCVLSRLSLERFSNFSANFVDNFLLVARFARGETLKNGIVLEISKICPKLYISPVPKLFDCSTDNLLFIYILFASKVRMKEMSTPTGLPALNGDVLSAINDMIGRVIIINTTDKKRYSGVLGAVSQDFDFGMQCVVEITKENENNLLRTESECRDKMVFHYSDIVDFAYVTQEIKKQHAVSKFVTDRQYHGDTPIEGEELQEWNGGEEDGLGGSIEDDVVVAGGQTAARRSNNHNNGTGWSVNDMFAANEKMNVVSTFKEDLTQYTTVEVVGTDEDRARAERLAREIESNSSSKFMANLENDDDERDLDKITRQEDFENGNGRKRNNNSFNQQQQQRRNPNIAPNGQPVNRRAEGLRGDRRNSGSSSANNSRYGAPAAAQQNYSQNQQQQQGQKGYRRQNEENDWQMAKGKGQNQGHDHSFRQQQKQMLDPRPNNNVKPADDKAQSATTATAAAGGSRVTDLKNWGNEFSIATAPKDQAPAVPAGNSGSAWNRGPPSSLVAKGSSNESTPPPTTNGEEAETKKEEAPSTSVDVAAAPVQNVQNDAEKHQEDDNVSVTSENDSVITSKSSSFKFNINAPEFKPRVAPATPTATTPVQNEYHPQQQPHPAMMAPQQGPPAPGMGMVPPHMGGPQNQGQPPMMMWQQTGQQQQGGGGYPQNHQFPIQHVPMQGVPGQMYGPGAATPVTVAQQPNQQHQVPTSAAGGQNHQLRDGEYREKQPLYMPYGPPQMVPVTSQQFYHSQYQGQMQQAAPYQMKMMPQQAPQGAYQQRYQQPQVYMMPPQGQQQQPRYQGPPPPQQQQQQQPQQQQFSGEQSRPQSHPNSQPTTPGPRGELPKMSGAPQNGNMQAESSSNASHSGSTSSQSGQRSGSPPGAVPPPPPPQQQHQQQQHPPHHAPPHVGAPPPQMMQQQQQHIQQYMQVFYPMIMPQQMPMQQNQHPQQSLMGERSDQGFPTSGYFDYRTMPNYQQQQQQQQQQMHRQNSLPQQFQGNQGVNPSGQQSGPPPPPPPSQQGTPRDQQHSQSPP

SEQ ID NO: 3 - DNA, 1230 bp, C. elegans
  CDS (1)..(1227)
atgcagatatttttcagactgagatctcgtaaagtatgccgaagtttgagaacagccgttgataaatttggaatctttttcgatggaattataatgccgttcaaagtgttcaattttccattgagcgtccgtagacaaattgtcagcgaattgaatatcctcgacgtgttcaacttctcagtttgctcaccaaaaacaaagtcaacaaccgaaaatatacaaaaagacgcgttcatcatgatgaaacatatttgtcagagaactgatgccggggaattgcataagataattatggaaaatgatgaatcgaatacgcatctcactgtacgtttcgagaaagttgacgagagccaggtcgggagatatggaaagctattcaaatgggttatagggaaaaatgctgttacatgctgcatttgtatcacatcaaatgaagaaattgtcttttactttaatggatcagcactgcaagatgttcatcaagaagtgtacaatgtgattgataacatccatatgactgcgaaaaatgaagttgcaattacactccaaaaaaacgaaactagacccagttgcctatcaccaggcgaatccgaatactcctgttgcctcccaaaattgaaaaatgctgatgaaatttcgatttttgcaccacggtctcggttggaacccggaattattatcaattatagcttccttgacgatgtattcttgaaatgtcccaattcacaagttgcacggatatatggtttgattgatagtaggattaaaaaagaatgctgcttgtttgatgtcaaggaacatgaatgtggagacttgggtccaatggcttatcgaattatgtccatgtttagaggacaacatttgtctttaaaggcagtcttaacaacagaacccgtgattagttacttgaaacactgggccagcggccggaaacatcagaatattgaaacactggaagtaggtgttgaaggatttgtttcgatgcgatacgctgggacaacaattcttgaagaattgaaagcaagaccttggaatggtgcgagaaggcctaggaattatattttttctaataaaacgcacggaaaaccgcagatcttcgattgtgtaaaatggtttgacattaaaggtgatgggaagctggcttcaattcttgtcggccattccgattttaaaatggttgtttggaatggctccgctacgagccttctcgctacttgtgtattgggttaa

SEQ ID NO: 4 - protein, 409 aa, C. elegans
MQIFFRLRSRKVCRSLRTAVDKFGIFFDGIIMPFKVFNFPLSVRRQIVSELNILDVFNFSVCSPKTKSTTENIQKDAFIMMKHICQRTDAGELHKIIMENDESNTHLTVRFEKVDESQVGRYGKLFKWVIGKNAVTCCICITSNEEIVFYFNGSALQDVHQEVYNVIDNIHMTAKNEVAITLQKNETRPSCLSPGESEYSCCLPKLKNADEISIFAPRSRLEPGIIINYSFLDDVFLKCPNSQVARIYGLIDSRIKKECCLFDVKEHECGDLGPMAYRIMSMFRGQHLSLKAVLTTEPVISYLKHWASGRKHQNIETLEVGVEGFVSMRYAGTTILEELKARPWNGARRPRNYIFSNKTHGKPQIFDCVKWFDIKGDGKLASILVGHSDFKMVVWNGSATSLLATCVLG

SEQ ID NO: 5 - DNA, 2754 bp, C. elegans
  CDS (1)..(2754)
atggcctctgctgttggaaaaggagtgaccgctttcaacaccatcaaaaacatcatttcggaactgaaaaaggtcgacaaaacattctccccgaattcggcacaatataaatacctcatggagcaaatgaaagccgaccaggttacaacaagaagatattcaaaagccgagaacgagtcagagagcgttgcaaaactctatttgagctatattaaaggaacccgacagcttaatgaacttcaagagagatataagggtggcgagaagagtgttgaagaggctgctgctattatgactaggaatgctcctccaggtcaagaatcgacagatttagcatggctcgtcacacccgcaaaagatcttgtcgagaacttctcaatcgacgtgttgaaagcactcgccggatacctcgaggttatcagacaagaaagtgaagacacagataatcaggtggatgcagcgacaacttacagattgttcgattttcaacgagcatgccggattattcaaggatcgtgtgccgtatatggtcgaaaagtagatcatgtctatgaactgacgatttcagttgtggatttagtcgagaacaaaggtcaagatgatggaaacaccggcagtagacgaggagctggaagacggaaaaattttaatttgggatcgacgaattacgacttggctgatatcgactcactgaaacaggaagctctagcaaattttgagaagacagtaaaagaagagaaaaaatcgattgatgcagtgagaatggtcgagaatgctgaagttatagagtctcaatatgagcgaaaaagctgtttagtagcaaagccaacccagttcatgttcaagctcaattacggccagctcaatcgtactgatgaacaaattttgaatgcaaaatctcgtcctgatgttatcggaaaagtaaaagattttgaaattaaaaagtcgaaagttaaacacgatcaacaaatactctactcccatgattgttatcgtggaaatttagaccaattcacactacctggtgctcgttggatgccagataataaggaattggcagctaattttggtgttgctgatttggaagttgagttagatttggagcaagagcatgagaagatcagtgcatatggaccgtttaaggatcctctctctggccgtgaagttgttccacctcctcgttggttcatagagcaagaagcagttcgtcaaaatcaagaaattcaatctcgtgccacatcgagagccatcacaattgcagctaaaactcttcgagattctcaaggctttggatctcaaccaactcgtctctcacaaccatttgttgaacgacatcgacagagtaatcatttgaatgatttcttgtcatttgtggagggaagagttaataagaatcgcccgagcactcatcttaccactggcttagttgatatgttcgttgataactttggatctgttatgcaaaacgatgaaccaaacactagtagaagacctgatgagaattacgcgcccatggactttgatgatgattttggtggtggaggagacgatgacgacgatgattatattcgaaatttgtccagaagggatgagaagcgagcaccggcaccatgggacgagctggataaaaatcatattatttggtacacgggagacgagaatctgccagtcgttagcaagccggtaaaaaagatcacgaaattccaaccaaaacccgctgaaatgttggctaggaagcaacgacgtgaggaaaagatcaataagtctcgacgagacgagttcatggaaactcacgactatctgcaggattactactattggagaagtgcggctcgtataaatccgattaaggactggaagatagaatcacttcgtactgctattctagccgagaagaaacgaagaatcaaggaaaagactgcaaaaatccgagaagctcgtatccagaatatgcagagaaaacggacagctcgtgtgatacccgtggagcaatttgaaccggtaacagaggatattccaacgtctaatagaagaaccctcggagctgaatatgacgacgtagtcgatgaagatcttgctgccgaagtcgagctctcaatgtttggaggtgggttcgatgacgacgaagaagacgtgcggccacgtggtgaacggcctcctatggctccaaacaacctagaattcgatgctcttcaaacagattttgatattccgcctgctgaatatgttccattaagattcgaggatattgatgatgccgaactgaacagtgttatcaatttacctggaaatttgctcatcgacaaagctttaccattgctcaagaagtttgctgaaaatcgaacagatcgcgaacaaatggcttacgagatggctaaagcatatgaagatgtggatgttgccgtgtcaactctacaagaacatgttgacaaatggcattctcgaatggaaccaatcctcgaagaaggagaaactcgaaaagagtatgatgttcacgcagttggtcgtgctgtcattggccaatacgatattgaaggtggcacgaaacgtttactagatttggttatggatcgtccatggtacgaaatcagtcgatacttcctttcgtgcctctttatgtgtaatgtcggaaatgtcatggtcagcgaggacatggaactgccactcgaggaaagaatcaactcgatgaaaatcacactactcaaaagagatatgcattgtgaaatgttcaaggaggctggcgctcttgatgcc

SEQ ID NO: 6 - protein, 918 aa, C. elegans
MASAVGKGVTAFNTIKNIISELKKVDKTFSPNSAQYKYLMEQMKADQVTTRRYSKAENESESVAKLYLSYIKGTRQLNELQERYKGGEKSVEEAAAIMTRNAPPGQESTDLAWLVTPAKDLVENFSIDVLKALAGYLEVIRQESEDTDNQVDAATTYRLFDFQRACRIIQGSCAVYGRKVDHVYELTISVVDLVENKGQDDGNTGSRRGAGRRKNFNLGSTNYDLADIDSLKQEALANFEKTVKEEKKSIDAVRMVENAEVIESQYERKSCLVAKPTQFMFKLNYGQLNRTDEQILNAKSRPDVIGKVKDFEIKKSKVKHDQQILYSHDCYRGNLDQFTLPGARWMPDNKELAANFGVADLEVELDLEQEHEKISAYGPFKDPLSGREVVPPPRWFIEQEAVRQNQEIQSRATSRAITIAAKTLRDSQGFGSQPTRLSQPFVERHRQSNHLNDFLSFVEGRVNKNRPSTHLTTGLVDMFVDNFGSVMQNDEPNTSRRPDENYAPMDFDDDFGGGGDDDDDDYIRNLSRRDEKRAPAPWDELDKNHIIWYTGDENLPVVSKPVKKITKFQPKPAEMLARKQRREEKINKSRRDEFMETHDYLQDYYYWRSAARINPIKDWKIESLRTAILAEKKRRIKEKTAKIREARIQNMQRKRTARVIPVEQFEPVTEDIPTSNRRTLGAEYDDVVDEDLAAEVELSMFGGGFDDDEEDVRPRGERPPMAPNNLEFDALQTDFDIPPAEYVPLRFEDIDDAELNSVINLPGNLLIDKALPLLKKFAENRTDREQMAYEMAKAYEDVDVAVSTLQEHVDKWHSRMEPILEEGETRKEYDVHAVGRAVIGQYDIEGGTKRLLDLVMDRPWYEISRYFLSCLFMCNVGNVMVSEDMELPLEERINSMKITLLKRDMHCEMFKEAGALDA

SEQ ID NO: 7 - DNA, 2169 bp, C. elegans
  CDS (1)..(2166)
atgcttggaatggaacgccaattggctggaaatatggctcaacggccacggagtccgtatacaattaatggaataccaactacgagggttcagccgattagcgccagcaatcagctttttactgcttccaatggcttcacacctccaaatccacatctccgaacaacttctcgtcgtgaagaatcaccatcttcctccgtatttccacataattcagattttgaacgtaaccgtggtggtgctccgaaccgtgctcaccagctccgccatccgaccacaagccacctattgaatacatctgtgacgtcatcgattcgggcgaatgttaattccaactcgaatacgccgattcattcgagaatcggaagtgctataactgaaaccgaatcaaatgtttgcgaatgcttgctctccgcacttcttggcatcgaaactcggcttttcactccaattcacagagaaatgaagatcacatcaactgcttcactgtcaacgacccatcaagcagtgtgctcacgtatcctaccaattgctaatacatatctactattatcacataaagagcccaacaacactggacaaatgactgaagctctgatggcggcgattcgacgaattattggtgattatatcactgatattgatgctttacgacggactagaggcatcaaattttatcaatgtcttccgcttattgatatgtggcaaaatcggctgagattgctgaaaatggcctataaaattagaaatttgccccaactggaacttctcgaaaacttgtacattctgcacgcggcgtatagctttgattcggatcaaaaatcaatacttgacgtgattcttgattatacattgggagttttttgtaatcaaatgatggaatggatgactacgggcgaaattcccaccagtgataagtggattattgagaagaatgagcagagcggagagctttttctgcggaaaattccaatttttatcacggattcggatgctagaattcttctcgaaattggaaaatcccttccacacgttgaatctgcatcagactcggatctggaggcaatcgataaagcgactacagtagtccgtactcaattgggtcccaaagcgattttctcgaaaattttgggccaaatcttgcgtattctccgtgatgttgtgtgtggaattgtgatgcggatggtgatggaaactggacgattaaaggaacatgtactcaaatcaacatcattcttctttttgagtgatcccagatttacgaccactctctacactattataaaagaagcatcacaaggacttcgtgttggaagtacaacgctcagccgtcaaactgtgtcaagtgcactggcagccgcactcgaatcgtgtcaatctacacaaaatattagtgataaggagaaaaaagtgaagaaattgatgttcacactggatgcgttgacttcgactggaagcacaccaaacgtgtcgtcaagaatgcaatttgtacaaccgttacgcccacgctacgcgcctcaaatggacctgatgaagcccattttttcagcctgcgacgatgcctacgaatcgatattccacgtaatttggtccatagatttggctcgttgttcaactcaagatgcttcttctcaaaatcttccagaaattcagcgtttcctcattaaaaactattctcttcgtgagaatgccacgtgtcttctgaatatgctcgcccacgtgttttccatcataaattccacgttgctccgtgtccgatgccacgtttctgtgcaattggctcgatttttggcccgttttttggcggcgatcgacgaaaaatgtgtcgacgtggatgacgtcattgctgagcaccggcgatttgtgcatcgtgcgacgtgtgtcgtttttctcaaaaattcggatcaaatcgaaaaagagctcgccactcttgtccgttccacgtttgatgctcaagaattcacgcaggaattcggagccacttggcgtgacgtcatcgagagcactggaaatgatgagaatgcgcggaaagcaagaattgcggagatttgtcagaaaaagacgattggggcgaggaatttgatggatagtgttaatacaactcaacgtaagctcaatgagctgctcgacgaacaaatcacctacggaaatgatgctcttttggattga

SEQ ID NO: 8 - protein, 722 aa, C. elegans
MLGMERQLAGNMAQRPRSPYTINGIPTTRVQPISASNQLFTASNGFTPPNPHLRTTSRREESPSSSVFPHNSDFERNRGGAPNRAHQLRHPTTSHLLNTSVTSSIRANVNSNSNTPIHSRIGSAITETESNVCECLLSALLGIETRLFTPIHREMKITSTASLSTTHQAVCSRILPIANTYLLLSHKEPNNTGQMTEALMAAIRRIIGDYITDIDALRRTRGIKFYQCLPLIDMWQNRLRLLKMAYKIRNLPQLELLENLYILHAAYSFDSDQKSILDVILDYTLGVFCNQMMEWMTTGEIPTSDKWIIEKNEQSGELFLRKIPIFITDSDARILLEIGKSLPHVESASDSDLEAIDKATTVVRTQLGPKAIFSKILGQILRILRDVVCGIVMRMVMETGRLKEHVLKSTSFFFLSDPRFTTTLYTIIKEASQGLRVGSTTLSRQTVSSALAAALESCQSTQNISDKEKKVKKLMFTLDALTSTGSTPNVSSRMQFVQPLRPRYAPQMDLMKPIFSACDDAYESIFHVIWSIDLARCSTQDASSQNLPEIQRFLIKNYSLRENATCLLNMLAHVFSIINSTLLRVRCHVSVQLARFLARFLAAIDEKCVDVDDVIAEHRRFVHRATCVVFLKNSDQIEKELATLVRSTFDAQEFTQEFGATWRDVIESTGNDENARKARIAEICQKKTIGARNLMDSVNTTQRKLNELLDEQITYGNDALLD

SEQ ID NO: 9 - DNA, 1578 bp, C. elegans
  CDS (1)..(1575)
atggacgtctcttattatgatggccctaaggatgaagtcatcgaagcaatgctgaaaagcgcggtgacggccatgaaattgggacaatacgaggatggaaaaggacgcttagaggacacaatggagttcggaacctccaattttcaactgcttggtacaatctacatgtattacggaagagtgtgcaggcatttgaaccacgacgccaaggccttggagtttttcgaacatgagttgaacatgttcaaattgatcttcaactacccagaagcatgtgattccacacgtcgcatcgtccagcaggcactcaaaatggaaaagttctccaaggctcgacggtttgctgaggatctcattgattacaccagcaataagaagaacggagagaagtatatcggtcaagctcgaattttgttcgcttccgtgtgcctcgaaggatgtgaaagagacgtcgagagtaatcaagatgagaagaagaagcttttgtcaatatgtgctgaacagattgcagccgtgaaattgttcaacgagaataatacggaaggagctgtgtctgagaccaaaattatgctaattgaggcgaaatgcttgtcactcgacgaaaaatacgaggaatcgcgtcgcaagtatcaagaatgcatcgattttgccatcaaaacagaccagtttgaagcagttcacatcgcctattacgacaaggctctctatgctgagacatatcttcttttctttattatcagagatctcagaagtgctctcttctacgccacgaaattcggaaaagagcgagatgtagtcaaatataagtcgaagctatccgaagagatgctgagaaatggcgaattccacgaagcatatctctacggattggaagcgcttgtatcgattcggaagcttggattgaacgaacacattggagatgtgttgcttacaatcgcaaagtgtctcattgcacttggaaaaagacgccaagctgcttattttatcatcttggggagtgttctgaccatcaaccaaagcagtttcaaactgttctacgagcagatcgacgtggcgatgaatcaagagagaagcgaaacggcaactgatcaagatgcatgcctcgcaattgattcgtctcctgatccgacatcctcgaatgacatgattaataagttcgtcgtcaaactggagcacgcaacaaatgttgaaacctgggaaatgattgtcaacggaatcattgaagaccagaagaaaccagtagcgatcgaaaagaaagagaacgaggaacccgtagacatgatggatctcattttcagtatgagctcgcgtatggatgatcagagaactgaactgtctgctgccagattcattccgcctcgtccagtgtcatcggcatcgaaaaagactacaaagagccacagaattctccctggactccgtgccaattggacaaaagtgcagtcgatgaagttcgatggtcacacaatgaataggatcctgaagaggtcgaagaaaagcaaatcgtcattggattctacaaattcgatacagggcgatgatactcgaagcgatgatgtgacaatgacgtccaaatag

SEQ ID NO: 10 - protein, 525 aa, C. elegans
MDVSYYDGPKDEVIEAMLKSAVTAMKLGQYEDGKGRLEDTMEFGTSNFQLLGTIYMYYGRVCRHLNHDAKALEFFEHELNMFKLIFNYPEACDSTRRIVQQALKMEKFSKARRFAEDLIDYTSNKKNGEKYIGQARILFASVCLEGCERDVESNQDEKKKLLSICAEQIAAVKLFNENNTEGAVSETKIMLIEAKCLSLDEKYEESRRKYQECIDFAIKTDQFEAVHIAYYDKALYAETYLLFFIIRDLRSALFYATKFGKERDVVKYKSKLSEEMLRNGEFHEAYLYGLEALVSIRKLGLNEHIGDVLLTIAKCLIALGKRRQAAYFIILGSVLTINQSSFKLFYEQIDVAMNQERSETATDQDACLAIDSSPDPTSSNDMINKFVVKLEHATNVETWEMIVNGIIEDQKKPVAIEKKENEEPVDMMDLIFSMSSRMDDQRTELSAARFIPPRPVSSASKKTTKSHRILPGLRANWTKVQSMKFDGHTMNRILKRSKKSKSSLDSTNSIQGDDTRSDDVTMTSK

SEQ ID NO: 11 - DNA, 1578 bp, C. elegans
  CDS (1)..(1575)
atggacgtctcttattacgatggtcccaaggatgaagtcgccgaagcaatgctgaaaagcgcggtgacggccatgagattgggacaatacgaggatggaaaaggacgcttagaggagataatggagttcggaacctcaaattttcaactacttggtacaatctacatgtattacggaagagtgtgcaggcatttgaaccatgatgccaaggccttggagtttttcgaacatgagttgaacatgttcaaattgatcttcaactacccagaagcatgtgattccacacgtcgcatcgtcgagcaggcactcaaaatgggaaagttccccaaggctcgacggtttgctgaggatctcattgattacaccagcaataagaagaacggagagaagtatatcggtcaagctcgaattttgttcgcttccgtgtgcctcgaaggatgtgaaagagacgtcgagagtaatcaagatgagaagaagaagcttttgtcaatatgtgctgaacagattgcagccgtgaaattgttcaacgagaataatacggaaggagctgtgtctgagaccaaaatcatgttacttgaggcgaaatgcttgtcactagacgaaaaatacgaggaatcgcgtcgcaagtatcaagaatgcatcgattttgccatcaaaacagaccagtttgaagcagttcacatcgcctattacgacaaggctctatatgctgagacagatcttcttttctttattatcagagatctcagaagtgctctcttctacgccacgaaattcggaaaagagcgagatgtagtcaaatataagtcgaagctatccgaagagatgctgagaaatggcgaattccacgaagcatatctctacggattggaagcgcttgtatcgattcggaagcttggattgaacgaatacattggagatgtgttgcttacaatcgcaaagtgcctcattgcacttggaaaaagacgccaagctgcttattttatcatcttggggagtgttctgaccatcaaccaaaacagtttcaaactgttctacgagcagatcgacgtggcgatgaatcaagagagaagcgaaacggcaactgatcaagatgtatgcctcgcaattgattcgtctcctgatccgacatcctcgaatgacatgattaataagttcgtcgtcgaactggagcacgcaacaaatgtggaaacctgggaaatgattgtcaacggaatcattgacgaccagaagaaaccagtggcgatcgaaaagaaagagaacgaggaacccgtagacatgatggatctcattttcagtatgagctcacgtatggatgatcaaagaactgaactgcctgctgccagattcattccgcctcgtccagtgtcatcggcatcgaaaaagactacaaagagtcacagaatcctccctggactccgtgccaattggacaaaagtgcagtcgatgaagttcgatggtcacacaatgaataggatcctgaagaggtcgaagaaaagcaaatcgtcattggattctacaaattcgatgcagggcgatgatactcgaagcgatgatgtgacaatgacgtccaaatag

SEQ ID NO: 12 - protein, 525 aa, C. elegans
MDVSYYDGPKDEVAEAMLKSAVTAMRLGQYEDGKGRLEEIMEFGTSNFQLLGTIYMYYGRVCRHLNHDAKALEFFEHELNMFKLIFNYPEACDSTRRIVEQALKMGKFPKARRFAEDLIDYTSNKKNGEKYIGQARILFASVCLEGCERDVESNQDEKKKLLSICAEQIAAVKLFNENNTEGAVSETKIMLLEAKCLSLDEKYEESRRKYQECIDFAIKTDQFEAVHIAYYDKALYAETDLLFFIIRDLRSALFYATKFGKERDVVKYKSKLSEEMLRNGEFHEAYLYGLEALVSIRKLGLNEYIGDVLLTIAKCLIALGKRRQAAYFIILGSVLTINQNSFKLFYEQIDVAMNQERSETATDQDVCLAIDSSPDPTSSNDMINKFVVELEHATNVETWEMIVNGIIDDQKKPVAIEKKENEEPVDMMDLIFSMSSRMDDQRTELPAARFIPPRPVSSASKKTTKSHRILPGLRANWTKVQSMKFDGHTMNRILKRSKKSKSSLDSTNSMQGDDTRSDDVTMTSK

SEQ ID NO: 13 - protein, 179 aa, C. elegans
MDEEKIVLIGNESDLLHCGYNFALRAGGKRYPSAAHYTHYQILMSLNLPETAVEELLCTNSCDVHQKALVILQENMPDGHTINSLAAYLSESLRSYSIHALQLRAESDKDFEKTLMKTNEALLIVCDEKNPQLGIGMNDKTFMEWMAKEKADSRQISYWMKNEHTKPADLGQNKLGQEI

SEQ ID NO: 14 - DNA, 3438 bp, C. elegans
  CDS (1)..(3435)
atgatgaaaaacgatgagcaggacgatgtggacgtggtgaatgtgccgacaaatatcgattttcagtgcaatttatgcgaatacgtcgggaaatccgatcgaagtctcagtgcacacaaaagacacagacatccacggccagaaggtcaggaagatgataaaaaaccaaggggccgaccgaaaaaatacgaagattctccggttccactgcaacgagtcaccacaaaacagcttctcggcggtcgaaaacgtccgaaaacgatagtggaatgtgtggatgattcggcaataagaaccgataaagaagcttctcgccttcacggactcggaattattgacgaagagacttcccttggggattatgttcgccgtggagccaattatcagctgatgggcggctcggacatgaatttagacggaaattccatgattgtcggccaagaagtggttattgatcatgaggaagacgaggaaaatgacgatagtcctatgttggattcgattcctgctggaaagaatctcgaaagacggaaaatacgtaaatccggaagagcatgtgcctcaatacgcttgtcagccaaaaaccgccaaatttccagatcttcacacatgcgaaaagtgcacccgaatgagcacgctgaacgtaagagaaatgctccaggatacaactcaaatggcacatacaactgtcattttccactttgtcagtggcggggatggtcccggtcgaccagaagcacacatttacgaaaagctcatcccgagtggcggcccgatgataatcgagtcatgacggttttgtcgtgtttctactgcaaaagctccttccacacgtacgctcagctggctgatcacacaataactcacggcggcttggcacttatgatttctcaaagtttcgacaataaacaccatttttcggcttggcttcaacaaatggaacgcgtctacagtgtcacattcgaacggtcggaatttggaagtgatcctgagctgcaggatcttcacaactatgtcctacactgcgattgcgtgggtggacgtggggagcagctcgccacggattcgagagcctacatcgaccaaaagcacacaaatttcaaattttccggagcctacatctaccaaaagcacaattttctgaaacgccgacacccacagctgctcactcggaataaaaatgactgttgtgtgcatttggaggtccgtgaggactcggaatacggcccgattcacgtgaaaggcactctggagcacacgggccatcgtttcggaactccgttgctcagaatgttattcgaaggcttcacaatggatcattacaccactccgcggtccgtaatcctgatggaaccccttcacacgaatcaattccaatccctcagagaattcatcgaggctccctccacctcaacctatcaaaatccgtatttttccgtcgattttggcgattcgccggagcaattttcgtttggttacatgtccgacgagatgcaaaagttgtggagcaaatatgcgcctggaaggacaattacaatcgattcgtccccgctggagtttggcgaagtggatttgttccagtacagcgtcattgtgaccgacgagaattacattccaaagtgtgcgatgatctacgtgacgacggacaattcacgggctccgcacacaattttgcggcaaatgaaggctgtagaacctcgaccgccgacaatttttatggtggacccgtcacaactctgggtggaacttatccaagacctatggcccacagatttggataatcccgagtcggaagtgacgattctcatctccgaatggactctgatggattattgggctgcgaaaacggaggaactagtcaaaaacgaatttgacgtgttttgtatcgttactgctctccgccgtatgctccgcgtcgaagatgctcatcaactctattacacaatcgtggaacttttcgaggcgtttttcgagtgcggctacgatggtttagccgaatttttcgacgctcagctctccgacatggaatattttaagcgttggggtccccttcatcggaccacactgacaagccacgcccatccgactctcggagcatctttcagaattctaagggattggtatttgaatccggcgaatatggatcacgacgcgccacgtgtagatcaatggttttcgcatatttgtcagcgaattgaggattttaatgcaattaccaatacacaaacctacactttgagacccttggaccccccgcataaattcttctatactacggtagaggatgaggagggcgaggaagctgagcacgaaaatccgttcaaaaacccgcaaattctaatgtacgacgacgacggggacgaggttgtcgtggaagaggaaatcatcggccacgaaattgacggcgccagcgagcttcaaggctcggagcttccagacgaagaagtgatctacgagacttatgaagattatcctgaatatggtggacattatgatctggaagaggagcataaaatgcaccataatatgcggttggaagaggaagagcttcagcaacatttacacgagcagcagaaacccgagcacctgagccaccaaaccgaggagctcggcgagcttcaggaccatcatcaccaccatcatcatcatctcgacgaggatccagaagccacagcagaaggaccatcagaaattgtggaacacgacgatgaggaggagcaggaggatcttgtcgatgtggaaatggtcgatgatcagcacgaacaacacgaaattccggcaaaaatcccgccgcaccggcaacggctctatcagaagctcgatccgatgcctgtaataagccatgagctgccgcggagcccgtcgcgaagaattcgaattcctacgagaaagatacaagaattggaccaagaatcagtgctcccaccaccaccgcggcgtgaaaccacccgctcagtagttcggagccgtaagcggcgagaccgattcgccgagctccgagactgtccgccagaggtgatgcgggccatcgccgcccacgcaatcgcctacgatggccggaaaaaagagcagcggccgattgtctatgtgcctaggcaggcgaaaatggcgatgagctcgacgccgactgctccgacgccgcccgaggattacgatgggcctcctggccatcaaatgacgacgattttacagcatcagagacaggcgaaacaggaggaagaggaagaacagtggagtggagagtatgatgatgagctccacgagcaagagcacgatcctgccacgtcatcaccgccaccgtcacttctaccacacacgggtccatcggaaattgtacaccacgaggaggaggttcatatgatgttaccggaggaagaagccgatctgatggacgacgatgatcaacagccatgtacatcggcttctatgtatcgctaa

SEQ ID NO: 15 - protein, 1145 aa, C. elegans
MMKNDEQDDVDVVNVPTNIDFQCNLCEYVGKSDRSLSAHKRHRHPRPEGQEDDKKPRGRPKKYEDSPVPLQRVTTKQLLGGRKRPKTIVECVDDSAIRTDKEASRLHGLGIIDEETSLGDYVRRGANYQLMGGSDMNLDGNSMIVGQEVVIDHEEDEENDDSPMLDSIPAGKNLERRKIRKSGRACASIRLSAKNRQISRSSHMRKVHPNEHAERKRNAPGYNSNGTYNCHFPLCQWRGWSRSTRSTHLRKAHPEWRPDDNRVMTVLSCFYCKSSFHTYAQLADHTITHGGLALMISQSFDNKHHFSAWLQQMERVYSVTFERSEFGSDPELQDLHNYVLHCDCVGGRGEQLATDSRAYIDQKHTNFKFSGAYIYQKHNFLKRRHPQLLTRNKNDCCVHLEVREDSEYGPIHVKGTLEHTGHRFGTPLLRMLFEGFTMDHYTTPRSVILMEPLHTNQFQSLREFIEAPSTSTYQNPYFSVDFGDSPEQFSFGYMSDEMQKLWSKYAPGRTITIDSSPLEFGEVDLFQYSVIVTDENYIPKCAMIYVTTDNSRAPHTILRQMKAVEPRPPTIFMVDPSQLWVELIQDLWPTDLDNPESEVTILISEWTLMDYWAAKTEELVKNEFDVFCIVTALRRMLRVEDAHQLYYTIVELFEAFFECGYDGLAEFFDAQLSDMEYFKRWGPLHRTTLTSHAHPTLGASFRILRDWYLNPANMDHDAPRVDQWFSHICQRIEDFNAITNTQTYTLRPLDPPHKFFYTTVEDEEGEEAEHENPFKNPQILMYDDDGDEVVVEEEIIGHEIDGASELQGSELPDEEVIYETYEDYPEYGGHYDLEEEHKMHHNMRLEEEELQQHLHEQQKPEHLSHQTEELGELQDHHHHHHHHLDEDPEATAEGPSEIVEHDDEEEQEDLVDVEMVDDQHEQHEIPAKIPPHRQRLYQKLDPMPVISHELPRSPSRRIRIPTRKIQELDQESVLPPPPRRETTRSVVRSRKRRDRFAELRDCPPEVMRAIAAHAIAYDGRKKEQRPIVYVPRQAKMAMSSTPTAPTPPEDYDGPPGHQMTTILQHQRQAKQEEEEEQWSGEYDDELHEQEHDPATSSPPPSLLPHTGPSEIVHHEEEVHMMLPEEEADLMDDDDQQPCTSASMYR

SEQ ID NO: 16 - DNA, 294 bp, C. elegans
  CDS (1)..(291)
atgatcaagtaccacgtgtcgcgaagcgcccgactctgggcttactggagcaccctgccacatcgaatgctcaagaaatacccggagcaaactatcttctacgcgactttcggagtagccacactattgattggcggatataaaatgaagaagtacttgacagaaagtgataagccattctaccgcggatactacgacgtcgtccgttccaacgacccaatcgcacaaaattggagaaaaccagaagaatatccagcaccgtatcttctttcaagtgttgagacagctcattga

SEQ ID NO: 17 - protein, 97 aa, C. elegans
MIKYHVSRSARLWAYWSTLPHRMLKKYPEQTIFYATFGVATLLIGGYKMKKYLTESDKPFYRGYYDVVRSNDPIAQNWRKPEEYPAPYLLSSVETAH

SEQ ID NO: 18 - DNA, 450 bp, C. elegans
  CDS (1)..(447)
atgacaactccaagtcctcaggaaatatcaactgtcacattgaactctttgtcgaaggaagaacgaaagaaagcaattctggccttacaaacaaagaaaaacgaaatcaaattagttattgatgaacatcagaagaaattagctgagtttcgacaatcgattcctgatgggtgctgtgaaatgttcgaaaacgaaaagaaaattcgtctgaaacatagttccacggccgaacttcttccacatttacaaaaagtaggctcgagcagtgtcgacaaagagaaaatgatcaacaacattcggcaaagagttgaagttcaagttgctgaccaggagacttttaagagtatcgaagaacggaatgaactgttgcgtcgagaaaacgagaatctcaaaaaagaagtcaataccgtggcagccgaactcctgcaagaatttgaaagttatgaataa

SEQ ID NO: 19 - protein, 149 aa, C. elegans
MTTPSPQEISTVTLNSLSKEERKKAILALQTKKNEIKLVIDEHQKKLAEFRQSIPDGCCEMFENEKKIRLKHSSTAELLPHLQKVGSSSVDKEKMINNIRQRVEVQVADQETFKSIEERNELLRRENENLKKEVNTVAAELLQEFESYE

SEQ ID NO: 20 - DNA, 1638 bp, C. elegans
  CDS (1)..(1635)
atgcagcagaatatacaatacacatacagttcggatccccgctcgggcggtaatcaaaatcagcagctacacggaatatcgcaatttcaacagaactcctatcagccgacgacgtattcaaattcaaatgcgaacaattttcattcctctaattccgcaacttttatacctaattcagtgacgcaaacacctcagcggcggatgggagaagagaagagtttagttaatatgcttgacgcttattttatcgacagtacgacaccttcaacatcaacgtcgtggaataatcagcagccagttcaggacctacaagtagaaggaatgaatgttaaactctcctcgtcacccgacaaaggcgagaaggatcgagtaaagaaggcgcgacaggcagaagccgcccgaatgcgttatcaccgactatcaaacgaagagaaacgagagttaaacctgaaaagaacgttggcgcagaagcgaaaacggcaacgtgagaaggagatggaggagctcgaatcgattttgcgggagacaaatgacattcaagaggatccggacatcaccgaacagctcagagagaagagaatgcgggccaagtgggcagaggccgcccgtacacgttacgctagaatgacgccggaagagcgccgggcgcacaatacgcggagacgtatgcggcagatgcagaacgcgatgagcgcgattaaggcatgtggaatggctacaggagatccacagaaggacgacgaggctgtccggttgcacatcaagacgcagaatgcgaaaaaagcggaggcggctcgtcaacgataccatcgaatgaacgatgacgagaagcgcgtctacaatcaacgaaggacagaagcgttccgccgccgtcgtatggaagaagagatgctgctcgccatgcctataggccgtatcaacggagaagcactggatcgtgctcagcagattgtggtccgaaacgcgaaacgagccgaagcggcacgtcttcgctaccagcgaatgacgcctgatcagagaaaggtcgcgaaacaagaaccgaaagaagagtcttggccctctcatgagcaatctgacacgcaaatcgaccatgatctcgatctcggagactcgtacaatcaaaaacgctacactccaaagcgtcgtcgcaacgagatggacgacatgatcagcgccggacagatggccgtcggatcctctggaaccatcacaatgggcaccggcacccccgccaaaaaggacgatatggacgctctctcctcgttggaacgtgacgtcgtcaagcgcacacagcacgctcagcagatccttctccgacaacgcgcgaatcagtcgtcgggaacgccacgctcggttacaccaccgccacaggcctatataatctcgaatggtcagcaacagcaccaacagttggttggaaatcagggaaacaatgggattggacagcaccaggtgctccatcaaggacacatgcaacagcagcagattcaggtgtcacaccagcagtcgtcccaatcgattcctcagaacatctacacttctggcatgattatccaaaatcagggacaacagcaacaaggccagcagcagcagcagggacagtctcaacaaatattgaacaatccttatagaacctaa

SEQ ID NO: 21 - protein, 545 aa, C. elegans
MQQNIQYTYSSDPRSGGNQNQQLHGISQFQQNSYQPTTYSNSNANNFHSSNSATFIPNSVTQTPQRRMGEEKSLVNMLDAYFIDSTTPSTSTSWNNQQPVQDLQVEGMNVKLSSSPDKGEKDRVKKARQAEAARMRYHRLSNEEKRELNLKRTLAQKRKRQREKEMEELESILRETNDIQEDPDITEQLREKRMRAKWAEAARTRYARMTPEERRAHNTRRRMRQMQNAMSAIKACGMATGDPQKDDEAVRLHIKTQNAKKAEAARQRYHRMNDDEKRVYNQRRTEAFRRRRMEEEMLLAMPIGRINGEALDRAQQIVVRNAKRAEAARLRYQRMTPDQRKVAKQEPKEESWPSHEQSDTQIDHDLDLGDSYNQKRYTPKRRRNEMDDMISAGQMAVGSSGTITMGTGTPAKKDDMDALSSLERDVVKRTQHAQQILLRQRANQSSGTPRSVTPPPQAYIISNGQQQHQQLVGNQGNNGIGQHQVLHQGHMQQQQIQVSHQQSSQSIPQNIYTSGMIIQNQGQQQQGQQQQQGQSQQILNNPYRT

SEQ ID NO: 22 - DNA, 1707 bp, C. elegans
  CDS (1)..(1707)
atgcagcagaatatacaatacacatacagttcggatccccgctcgggcggtaatcaaaatcagcagctacacggaatatcgcaatttcaacagaactcctatcagccgacgacgtattcaaattcaaatgcgaacaattttcattcctctaattccgcaacttttatacctaattcagtgacgcaaacacctcagcggcggatgggagaagagaagagtttagttaatatgcttgacgcttattttatcgacagtacgacaccttcaacatcaacgtcgtggaataatcagcagccagttcagatgtcttcaatacaacaatcagttggatcatacaatcaacaaatacctcagttgaaagtaggagatgtggacctacaagtagaaggaatgaatgttaaactctcctcgtcacccgacaaaggcgagaaggatcgagtaaagaaggcgcgacaggcagaagccgcccgaatgcgttatcaccgactatcaaacgaagagaaacgagagttaaacctgaaaagaacgttggcgcagaagcgaaaacggcaacgtgagaaggagatggaggagctcgaatcgattttgcgggagacaaatgacattcaagaggatccggacatcaccgaacagctcagagagaagagaatgcgggccaagtgggcagaggccgcccgtacacgttacgctagaatgacgccggaagagcgccgggcgcacaatacgcggagacgtatgcggcagatgcagaacgcgatgagcgcgattaaggcatgtggaatggctacaggagatccacagaaggacgacgaggctgtccggttgcacatcaagacgcagaatgcgaaaaaagcggaggcggctcgtcaacgataccatcgaatgaacgatgacgagaagcgcgtctacaatcaacgaaggacagaagcgttccgccgccgtcgtatggaagaagagatgctgctcgccatgcctataggccgtatcaacggagaagcactggatcgtgctcagcagattgtggtccgaaacgcgaaacgagccgaagcggcacgtcttcgctaccagcgaatgacgcctgatcagagaaaggtcgcgaaacaagaaccgaaagaagagtcttggccctctcatgagcaatctgacacgcaaatcgaccatgatctcgatctcggagactcgtacaatcaaaaacgctacactccaaagcgtcgtcgcaacgagatggacgacatgatcagcgccggacagatggccgtcggatcctctggaaccatcacaatgggcaccggcacccccgccaaaaaggacgatatggacgctctctcctcgttggaacgtgacgtcgtcaagcgcacacagcacgctcagcagatccttctccgacaacgcgcgaatcagtcgtcgggaacgccacgctcggttacaccaccgccacaggcctatataatctcgaatggtcagcaacagcaccaacagttggttggaaatcagggaaacaatgggattggacagcaccaggtgctccatcaaggacacatgcaacagcagcagattcaggtgtcacaccagcagtcgtcccaatcgattcctcagaacatctacacttctggcatgattatccaaaatcagggacaacagcaacaaggccagcagcagcagcagggacagtctcaacaaatattgaacaatccttatagaacctaa

SEQ ID NO: 23 - protein, 568 aa, C. elegans
MQQNIQYTYSSDPRSGGNQNQQLHGISQFQQNSYQPTTYSNSNANNFHSSNSATFIPNSVTQTPQRRMGEEKSLVNMLDAYFIDSTTPSTSTSWNNQQPVQMSSIQQSVGSYNQQIPQLKVGDVDLQVEGMNVKLSSSPDKGEKDRVKKARQAEAARMRYHRLSNEEKRELNLKRTLAQKRKRQREKEMEELESILRETNDIQEDPDITEQLREKRMRAKWAEAARTRYARMTPEERRAHNTRRRMRQMQNAMSAIKACGMATGDPQKDDEAVRLHIKTQNAKKAEAARQRYHRMNDDEKRVYNQRRTEAFRRRRMEEEMLLAMPIGRINGEALDRAQQIVVRNAKRAEAARLRYQRMTPDQRKVAKQEPKEESWPSHEQSDTQIDHDLDLGDSYNQKRYTPKRRRNEMDDMISAGQMAVGSSGTITMGTGTPAKKDDMDALSSLERDVVKRTQHAQQILLRQRANQSSGTPRSVTPPPQAYIISNGQQQHQQLVGNQGNNGIGQHQVLHQGHMQQQQIQVSHQQSSQSIPQNIYTSGMIIQNQGQQQQGQQQQQGQSQQILNNPYRT

SEQ ID NO: 24 - DNA, 1620 bp, C. elegans
  CDS (1)..(1617)
atgcagcagaatatacaatacacatacagttcggatccccgctcgggcggtaatcaaaatcagcagctacacggaatatcgcaatttcaacagaactcctatcagccgacgacgtattcaaattcaaatgcgaacaattttcattcctctaattccgcaacttttatacctaattcagtgacgcaaacacctcagcggcggatgggagaagagaagagtttagttaatatgcttgacgcttattttatcgacagtacgacaccttcaacatcaacgtcgtggaataatcagcagccagttcagatgtcttcaatacaacaatcagttggatcatacaatcaacaaatacctcagttgaaagtaggagatgtggacctacaagtagaaggaatgaatgttaaactctcctcgtcacccgacaaaggcgagaaggatcgagtaaagaaggcgcgacaggcagaagccgcccgaatgcgttatcaccgactatcaaacgaagagaaacgagagttaaacctgaaaagaacgttggcgcagaagcgaaaacggcaacgtgagaaggagatggaggagctcgaatcgattttgcgggagacaaatgacattcaagaggatccggacatcaccgaacagctcagagagaagagaatgcgggccaagtgggcagaggccgcccgtacacgttacgctagaatgacgccggaagagcgccgggcgcacaatacgcggagacgtatgcggcagatgcagaacgcgatgagcgcgattaaggcatgtggaatggctacaggagatccacagaaggacgacgaggctgtccggttgcacatcaagacgcagaatgcgaaaaaagcggaggcggctcgtcaacgataccatcgaatgaacgatgacgagaagcgcgtctacaatcaacgaaggacagaagcgttccgccgccgtcgtatggaagaagagatgctgctcgccatgcctataggccgtatcaacggagaagcactggatcgtgctcagcagattgtggtccgaaacgcgaaacgagccgaagcggcacgtcttcgctaccagcgaatgacgcctgatcagagaaagtcgtacaatcaaaaacgctacactccaaagcgtcgtcgcaacgagatggacgacatgatcagcgccggacagatggccgtcggatcctctggaaccatcacaatgggcaccggcacccccgccaaaaaggacgatatggacgctctctcctcgttggaacgtgacgtcgtcaagcgcacacagcacgctcagcagatccttctccgacaacgcgcgaatcagtcgtcgggaacgccacgctcggttacaccaccgccacaggcctatataatctcgaatggtcagcaacagcaccaacagttggttggaaatcagggaaacaatgggattggacagcaccaggtgctccatcaaggacacatgcaacagcagcagattcaggtgtcacaccagcagtcgtcccaatcgattcctcagaacatctacacttctggcatgattatccaaaatcagggacaacagcaacaaggccagcagcagcagcagggacagtctcaacaaatattgaacaatccttatagaacctaa

SEQ ID NO: 25 - protein, 539 aa, C. elegans
MQQNIQYTYSSDPRSGGNQNQQLHGISQFQQNSYQPTTYSNSNANNFHSSNSATFIPNSVTQTPQRRMGEEKSLVNMLDAYFIDSTTPSTSTSWNNQQPVQMSSIQQSVGSYNQQIPQLKVGDVDLQVEGMNVKLSSSPDKGEKDRVKKARQAEAARMRYHRLSNEEKRELNLKRTLAQKRKRQREKEMEELESILRETNDIQEDPDITEQLREKRMRAKWAEAARTRYARMTPEERRAHNTRRRMRQMQNAMSAIKACGMATGDPQKDDEAVRLHIKTQNAKKAEAARQRYHRMNDDEKRVYNQRRTEAFRRRRMEEEMLLAMPIGRINGEALDRAQQIVVRNAKRAEAARLRYQRMTPDQRKSYNQKRYTPKRRRNEMDDMISAGQMAVGSSGTITMGTGTPAKKDDMDALSSLERDVVKRTQHAQQILLRQRANQSSGTPRSVTPPPQAYIISNGQQQHQQLVGNQGNNGIGQHQVLHQGHMQQQQIQVSHQQSSQSIPQNIYTSGMIIQNQGQQQQGQQQQQGQSQQILNNPYRT

SEQ ID NO: 26 - DNA, 1524 bp, C. elegans
tgtgcgttttaatttatgcctacgtgcctgcctaccacctatcttctgcaatttggagtttcgactgtggagatacgtttgaattggacttatccccaaagatgaattatcaaaacgaacaaaacgcctatcaaatgcagaatcaggaaaatgttccaccgatgagttacgggtacccaatgccaactccggagcaattccaaaaaatgagttacgggcagaacttccaagctccatgggatatgcagcagcagattcagatgtttcagcaatatcgccaaaatttccaaggaggatattcggacccattcacctaccagaatcagtatgcacatcagccaaaaattgtgacggtcactttgcaggacccacaggacctctggaaggagcttcattatttgagtaatgagcagaatgtgcttaataatgggaggaaaatcttcccagccctcaactacaaagtcgagcatctcaatccagaatccaactacaaagtggaaattttgctccgccgaatggtcccctatcagattcaatattcaaatggaagctggtcccggaagaatgtacagagcaagaagactattgcaatgaaaacagaaaaagtatttgttggagagttcactggacaagatatcatgcgtaccggcttggatttatcagatgtgaaggtgtttaatattggaagtgataataagaagaaagtgactccatatgaagagatgagtgacgcagagaaacgagagtatgatactcagtattcgaagaagaagacctccatggtaggtactgccaaaatgtgggcgggcgtaatcttaagccgaagcctgattcaaagcctaacccgaaccctaagcctaggcctaagccaaagccaaagcctaagcctaaccctaagcctaaccctaagcctaaacctatacctaatcctaaacctaagcgtcgaggtatcaaatgagtgcaagtacatcccggtactcatcatcaacgagattctaccaaatcaagagcttcgattggttggggaatttgagaacgagattacccaatttgcgacagtttcttcatacaaggaccacatcgtaaaagccttgcgcacagccgccaacccaacttctcgcggagacgccaaacaggaagccgttcaggtcggaaagcaatggagatcatccaacaagtccttgctggaatccctcagaccatctatgatttgcaccagtgtctccacaactgctccaagcaccaatttccatgctccactgcagtaccccggcacctcatccccatccagcaactttgctccaatgacaccttccacgtcattcgactcggcgtacagttccttcaacgtgacgtcatcaactccagaacaaatgtgctacaatccgattccttcaatgtcaactgactactcattttgctcattcgactctgcgacatcatcaccgccacttcagccaacagccacgtcaccagaggcttctcaaaatcagattaaacttgaaatgaaccaatacatgtaat

SEQ ID NO: 27 - DNA, 702 bp, C. elegans
  CDS (1)..(699)
atggctcttctcctagtgatgggcatcccagcttctggaaaatccacatttcgacgaaaaatccaaactgctcaccgggagcttctcgaatccacgtcatttgacgatttccgtatgatgcgaacgcgctccagtgataaaaatgcgcgccaaactcgaaaatcgttcgagtgtcacgtggaatcgtgcatttccgaatctacatcgaaaatttacgtcatagaagacattttctatctgaaaagtaaacacggacaaacacatttagttttacaagccgcgacgcgacacgcaacgcgccgtaaatctaccccagatatggccgagccaaaatggcctagttcggcaaactcttccatttcaatttatgagggaagccagaaatccgtggacgtattatcgcggaaaattccgaaatcgagatgcaatcgcgctccacggacaaaagtctcgttaggtggcaaaaccagatattcgaagaaacctccaccaccatcaaaactcgaatctctggatgttctaacccgtcgattggtgtctgaactaatgcaaaatgataaaacaatgaatggtcgaaaactcagtgatgctaggaagaagcttctgatagatttatctggaaatttggaaatttcaaatgaaaacgatcccaaattcttatatgatctcaaattgaaattaattgaattttatgataaataa

SEQ ID NO: 28 - protein, 233 aa, C. elegans
MALLLVMGIPASGKSTFRRKIQTAHRELLESTSFDDFRMMRTRSSDKNARQTRKSFECHVESCISESTSKIYVIEDIFYLKSKHGQTHLVLQAATRHATRRKSTPDMAEPKWPSSANSSISIYEGSQKSVDVLSRKIPKSRCNRAPRTKVSLGGKTRYSKKPPPPSKLESLDVLTRRLVSELMQNDKTMNGRKLSDARKKLLIDLSGNLEISNENDPKFLYDLKLKLIEFYDK

SEQ ID NO: 29 - DNA, 738 bp, C. elegans
  CDS (1)..(735)
atgaatcaaaaaccgacgcctttgatgtcaccgcctccgcctgctcaacaaaacggaaaagatcacaaggaaaagctccatcgcattgaggacatcatgtattcggacgccgacacgtttcccgaattttcgaatcgtgatggatcacgtgaaacggtggacacttgttggcagctagtcttgattcgtgtacccgaacatccaaatgagactcacatcaaaacaatcaatgacgcaattacgagtgagatgaaagccacgtggaagatcccggccacttcgttcgcttacgtctccgcgccacacattccgattttgcagttggcttacggagttgcgaagaagcattttggggagaagaccgcgattcgatcaagcttcatgtttgcagagccgaagaacgataagtttctaatcgctaaggattcggacttgaagcatctcaaatatgaggtgatccttggcgcgagccgcttctgttggacaaaagtggagactcacgcggaaatgtcaaaacgaattgagtcgacactttttgaattgatgcgtgttcttccgaatcttgtccttttcctcggaccgctcgcgtttgacgttctctacgagacactcgagatgcagaaacgaaaaacgattgccaaaccgggagtagtcatcgagaatttcaaacgggaaaataacaaatggaagcgtgttcagaagcacggagccacgtcgatctctgttccattgtgttga

SEQ ID NO: 30 - protein, 245 aa, C. elegans
MNQKPTPLMSPPPPAQQNGKDHKEKLHRIEDIMYSDADTFPEFSNRDGSRETVDTCWQLVLIRVPEHPNETHIKTINDAITSEMKATWKIPATSFAYVSAPHIPILQLAYGVAKKHFGEKTAIRSSFMFAEPKNDKFLIAKDSDLKHLKYEVILGASRFCWTKVETHAEMSKRIESTLFELMRVLPNLVLFLGPLAFDVLYETLEMQKRKTIAKPGVVIENFKRENNKWKRVQKHGATSISVPLC

SEQ ID NO: 31 - DNA, 1812 bp, C. elegans
  CDS (1)..(1809)
atggctgatggaccagataaaaaacgcagaaagctgacaagtgctctcaaagacgcaatcggagcgcactcgcaaaaacctgaggaaaacatagaaaaaccaattgctctcgcacttattagcgacaacaaggccgcgcaagaccttctcaacaaaattcaagccagtcggcgcccgcaagcgattttggaggcaactttgaagatctgtaaggagaagaaatccgaagcgcacgttgatgaagcgtttctttcaattttccgcaaacatttctcgattctcggtgtggattctgccttggcatgctggaaattgatcgaagaacatctcaattcttcagaatcaataaacgaaatcagaattgctaacgagttcatcagtacctctaatcttcttggccatggtttcggaaaacttctgcaaaacatgaagcaaggaattcctgtggtaattcagaatacgtggagacaagaacgtaaagcggacgccttagctcttgcctatcccttcctaagcctcctcctagctgatcaggtatatggagaccatcaaaatcttgaatctttggcagaattctggaaaaatcaagttttaccggagaaatccatgaaaaaagattttggaatgagaatggtcagtgctttagccggcggtgagcctggacagttgccagaaacgaaaatcgacgagaatcaacttgaaatgctctcaaagatctacgaaaatcatctggaattctacactggaattctctcagaatccacgtgctctcagcttcttgccaaagtaattgaggataatttagccaaaaatgagctaaagcaacttgcccgtcgatgccaaactgctgaagtatgcatgtccccactaaaagaaatcacaaagcatctcccttcaatcatcaattcctgctccaacactcgccaaattaccgctagcaagaagcttaaaaatctcgaaaatcttcaggatttccttcacaatgtcacagaaaaaccggaaaacgacaatttctcggctgcttttgcccttctttcaatcaatatttcactaatcttcgataactggaacttttgcgagaaaatcctgaaaactctaccgaaggacgttctggaaaagctgataaaagcaggaaagggatctgaaaatgagaatattttgaaggttttcaagaaagtggctcaggaagctgctccagtcaaatttgtgaatagaaccgccgcggatgtcgctgagaagctccgagagccggaatttgcagtggcagaagccttaagcttgctccaaggagttcatacagcggagacgaaaaaggaagtctttgattctgttgtaaaattcgtcgaagagattcctggagccgagagcattgacgagatgaagcttttcaagttgataattgaaatgtatggtggaagtcaactggaatcattagctgttcaagtagtgttttcacgaattgctgccgacggttggactgaaatggatcgggctccgggaaccgagaaatctgatgaatcatggtttacagtacttgtggagacgattgatctgcttgatatagcaatgaaatcggcgaagaattcagtgctaaaatcattcactgcacttttctgtcaactttttagccaagttttgaagaattctcaaaaattcgtgaggaatagtctgaacgcggtcgcgtcgcgtacactttcgcctgaaatggaaactgaatttgtgctccgccgtcataaattgagtcaggatttggcaagattagtagatgcaatgaagaggaatgaatcgtattttgcaatggtaagattttga

SEQ ID NO: 32 - protein, 603 aa, C. elegans
MADGPDKKRRKLTSALKDAIGAHSQKPEENIEKPIALALISDNKAAQDLLNKIQASRRPQAILEATLKICKEKKSEAHVDEAFLSIFRKHFSILGVDSALACWKLIEEHLNSSESINEIRIANEFISTSNLLGHGFGKLLQNMKQGIPVVIQNTWRQERKADALALAYPFLSLLLADQVYGDHQNLESLAEFWKNQVLPEKSMKKDFGMRMVSALAGGEPGQLPETKIDENQLEMLSKIYENHLEFYTGILSESTCSQLLAKVIEDNLAKNELKQLARRCQTAEVCMSPLKEITKHLPSIINSCSNTRQITASKKLKNLENLQDFLHNVTEKPENDNFSAAFALLSINISLIFDNWNFCEKILKTLPKDVLEKLIKAGKGSENENILKVFKKVAQEAAPVKFVNRTAADVAEKLREPEFAVAEALSLLQGVHTAETKKEVFDSVVKFVEEIPGAESIDEMKLFKLIIEMYGGSQLESLAVQVVFSRIAADGWTEMDRAPGTEKSDESWFTVLVETIDLLDIAMKSAKNSVLKSFTALFCQLFSQVLKNSQKFVRNSLNAVASRTLSPEMETEFVLRRHKLSQDLARLVDAMKRNESYFAMVRF

SEQ ID NO: 33 - DNA, 951 bp, C. elegans
  CDS (1)..(948)
atgacccaagaaccgtcagacaccagtgattatcaacgatttgatgaaatgcttcttcatttggagccaagaggggcaagagttccttataacgttgtttacgaccaagacaacaatatatgggtagcttcaaaaggcggtttatataaattcaacggaaaaactctccgaactctgcacgaagacaagaaattcttcaagaaaatggccccatttccacaagtaatttcgtacaaagacaggataatctacacatcagctgaattctctgatcgaacgacaattttaagagtaatttcacttgatggagatgttgttcatgagtcgtttatcgaaggattattaaattccatgacaatcactgatagtggcgatatgtatattgtgaagcaagctgaaccaggacatcgaaacaattttatcatgacaacacacttggacactccaatcggttgggatgttgttgctgaatcaaagcctggagaagctttttcacgaatttgtgcacttgatgacaagactttggtggcttcaatcaacgattttccaatgaatatgttctcaaaacaacgacttgtattctacgatttggaaacaaatactgaaacaggaagcacatcgaaagcaggaaaagagcccggagagatatttttcccgagattcatcaaaaaatatggagatggattcatcctaaacgacaaatctggacgattctcagagttcaagaaaaatggggaattcgtggcaattcgtgctcgaatcgatgcatttttgggcgaaggatttgatgtaaaagacggagaagctctgatggcattgactggaatggttaaagatccaaatgaacaattaatttgtgatgattggttagaaactataaaattggacggatcctcgtggaaagcagatagagaaaagaagaaacaagaagacgagcaagcgaaaaattga

SEQ ID NO: 34 - protein, 316 aa, C. elegans
MTQEPSDTSDYQRFDEMLLHLEPRGARVPYNVVYDQDNNIWVASKGGLYKFNGKTLRTLHEDKKFFKKMAPFPQVISYKDRIIYTSAEFSDRTTILRVISLDGDVVHESFIEGLLNSMTITDSGDMYIVKQAEPGHRNNFIMTTHLDTPIGWDVVAESKPGEAFSRICALDDKTLVASINDFPMNMFSKQRLVFYDLETNTETGSTSKAGKEPGEIFFPRFIKKYGDGFILNDKSGRFSEFKKNGEFVAIRARIDAFLGEGFDVKDGEALMALTGMVKDPNEQLICDDWLETIKLDGSSWKADREKKKQEDEQAKN

SEQ ID NO: 35 - DNA, 291 bp, C. elegans
  CDS (1)..(288)
atgaaccgtaccgctttcaaccgcttcttggctcttagtcgttttgcacaaaagcgtcacgggcacggacatcacgtcgccaacccgggaccaccatgcaccttcgacttgatgccagtgccattccaatcttacaagaaggtctacggcgagctccagggaaagttcaacacctatcttgcagtgtcagcggttttcttcgcttcgtcgttcgccaccgccatctacactaacctcttcgccttcgaagaagccatcagaccaccacagagctatcgcaatcgcaaataa

SEQ ID NO: 36 - protein, 96 aa, C. elegans
MNRTAFNRFLALSRFAQKRHGHGHHVANPGPPCTFDLMPVPFQSYKKVYGELQGKFNTYLAVSAVFFASSFATAIYTNLFAFEEAIRPPQSYRNRK

SEQ ID NO: 37 - DNA, 1041 bp, C. elegans
  CDS (1)..(1038)
atgcaggcagacggagaaaaaaagaagaaaaaaactaaccccgagaggagtacacacgacgatactccgaaatcccgcacccgcgtgttgtttagccaatatttctttttaagtttttcgctgttttttcgagcaattttcatgctgagaagtctgtgctcaatagctgttcgacttggcggtgcaagacaacctcgattgctgtccagcgcagcttcgggcgatggaaatgatggtaaaggtgcaaaagacgcgattgatgaggatttgctgaatgcgattgaaggagtagcgaataatattcacccgcagaatggctccgaaaagaaatcactaaaaaatacattgatcaatcgattagtagcaaacgaaaaagcgagttttgatgctgccgcagcttctgcaagcagtgaaatgctcgatgatcaggcacttatcggacttcttgccgatgtggcgggcgatgccaaagttgagaagaagcttcctcccaaatccgctcaacttcgacaagaaaaacgaggacttgttcttctcagaaaagagattttctatcaggcagttcaaagtggcttcacgacagaagaagctcgtgtcaaatcagagacaattgtcaatgaagcacagataaagctccaagagcagcgaaaagctcttctaaatgatgttagagaaaaggtggaacaagaagaagttgaagagacggagagaagtgagaaggatcagaagttgttcacgatggccttggaatttatggaaaagatttacaaggatgatttgattagtagtgctgttcaatttccaacggctcattcggaccaacaaattctttcgaagaacaaatcgaatggacaacaaaaggaaaacaatgggaatatccaatcgataatgagttcaaaatgggcgatgaatcgaatgtttcattcattgatcacgtattcttggagagacatttaccatcattgggtatcccgaaatctggtccaattgctcattttatgcatttggtttgtgttggtttatccaagaatccatatatga

SEQ ID NO: 38 - protein, 346 aa, C. elegans
MQADGEKKKKKTNPERSTHDDTPKSRTRVLFSQYFFLSFSLFFRAIFMLRSLCSIAVRLGGARQPRLLSSAASGDGNDGKGAKDAIDEDLLNAIEGVANNIHPQNGSEKKSLKNTLINRLVANEKASFDAAAASASSEMLDDQALIGLLADVAGDAKVEKKLPPKSAQLRQEKRGLVLLRKEIFYQAVQSGFTTEEARVKSETIVNEAQIKLQEQRKALLNDVREKVEQEEVEETERSEKDQKLFTMALEFMEKIYKDDLISSAVQFPTAHSDQQILSKNKSNGQQKENNGNIQSIMSSKWAMNRMFHSLITYSWRDIYHHWVSRNLVQLLILCIWFVLVYPRIHI

SEQ ID NO: 39 - DNA, 1677 bp, C. elegans
  CDS (1)..(1674)
atggtagtcgaaggatatgaggaaattgcagtggatgacgatatgaatgatatgagagaaatggttgcaaatgatgaagatgacagtattcactatgaagcccatgatggaagcgaatatggtggaatgccaattgaccaatatcaagatattgttatgttcaaaacaacggaccaagttcctgcaagtgtctcctctcgtcttgatcgtagtgcgccgagtcattggagagctcgttgtaaatgtttttcaatggcggacgatagggaaactcttttgtattacaatcccgagaagtctgcgaatactattccaaaagtagtagtgaagaaaggagaagttcaaaaagtccttgagagaattcacgaacttattggtcatcttgggcagaaaagaacgcagatggtcgtgcttcggaaactctactggcggtcggttcgacaagatgttaaaacatttattgcctcttgtgatttttgtacagcaaagaaaatacaaggcagaaaaataacaaaagcacctgtggatattacctctgagcattttgatatttccgtgcttgttagagattcttccagcggaaatgatcgatttgagtttcaattggtgggatacaatgaggatgaagttcgcgaagcatcatttacaaggatgacatcatatacattcaaagaaactgagcatgcattcagaagcagatacagtaaccatcagcagactgggccgccaacgttccgtcgacaaccatatgtgaagaaacacaaaaatcaatcagttggatatcttcttccatttcaccaaagatctcgtgccacagaacccgagtttatcgaagtttttgggcgcgacatgttcactcatcatcctgatatgatgtatgaaacagttccagaaatggacgatggaatgatgagatcaagagaagaagttaaaagaaaagatgaagaactgcccaatgcacatccagtaactgttggcggagcagtactgaactcgaataatttcaaaacggaagatgagggaaatcttgctgtctcttctcgttctcaagaatcgacgtcttcagcatcaccacaaaaaatgaaccatgaacagcaaagatatagtcaaaaacgtcatttggagatgagaactggaagaacgacgagagatatgagagattatgagtcatctggacgacaattatcaccaactccaacgactacagctggacttttgaaaaagagaaggcgagagttgccaagtattcgaggaatggcaaatcgtttcagtctggcaatcggcgactcggatagaatagataacaccgaagtaaatcgctcaaatccacatctgatcgagtaccctgatccttcgtctattcttcgtggtgataatgcaggattgcctcccgttattatggcgccatccaccaatgatgaagtatgcaaactgcagatcgaggcactccaacgtcatattcatctacaaaaaatgcaagaaaaactactccatgaacagtatgaagcttccatgcgaattccaatcacccgatacatcgaacaagaagaggaagttcaggaagaacaactcgaagttgagcatcatcatgtcatggaaaatcatcaccaagtgtacgaggaagtagaggaagaagtggttccgcaaaatccccgtcggcacattcgtcaccagtag

SEQ ID NO: 40 - protein, 558 aa, C. elegans
MVVEGYEEIAVDDDMNDMREMVANDEDDSIHYEAHDGSEYGGMPIDQYQDIVMFKTTDQVPASVSSRLDRSAPSHWRARCKCFSMADDRETLLYYNPEKSANTIPKVVVKKGEVQKVLERIHELIGHLGQKRTQMVVLRKLYWRSVRQDVKTFIASCDFCTAKKIQGRKITKAPVDITSEHFDISVLVRDSSSGNDRFEFQLVGYNEDEVREASFTRMTSYTFKETEHAFRSRYSNHQQTGPPTFRRQPYVKKHKNQSVGYLLPFHQRSRATEPEFIEVFGRDMFTHHPDMMYETVPEMDDGMMRSREEVKRKDEELPNAHPVTVGGAVLNSNNFKTEDEGNLAVSSRSQESTSSASPQKMNHEQQRYSQKRHLEMRTGRTTRDMRDYESSGRQLSPTPTTTAGLLKKRRRELPSIRGMANRFSLAIGDSDRIDNTEVNRSNPHLIEYPDPSSILRGDNAGLPPVIMAPSTNDEVCKLQIEALQRHIHLQKMQEKLLHEQYEASMRIPITRYIEQEEEVQEEQLEVEHHHVMENHHQVYEEVEEEVVPQNPRRHIRHQ

SEQ ID NO: 41 - DNA, 687 bp, C. elegans
  CDS (1)..(684)
atggagctgagcccgaatttgaaaagtaacttgaacagcgaaatgaacgatttggtacaacaagcagaagaatgggctcaatctcaacatcgttcagcttctcctggtgcattgaaagatccgaaactttgtgcaatttacaaaatgattctacaaaattcggctctacttttaatcgaaaaatctggtgtaaaaactgtggaagagtttcaaaaagtattcggaccaattattgaagaacagaaaaacaaagaagatgagattatcgaatttgaggaattcgaggaaattgagaaagcccgagcagaactagcttctccaacttcatttacaaaatattcgatgatgctcgacgagccattgtctgcgtctcctttcaatcgtcgccgaacctctttgttctcggatcttcatgtactgtcaccaattgcttcagcatctagtgaatcttttgagaaacctaaaagatttgcttcgtccactcaacacgctaaaaaatcttttgtcacagtggaatctactagaactccgtggagaagatcaaactcgatgtatctggaagatgtgctcaacagaacaattaatacgcttcttcctcccactgaagtcccacagtgcttcaagactgaatcatttgacaccatcacctcgtcctggagaaacgccattggagaatcgagcgagtag

SEQ ID NO: 42 - protein, 228 aa, C. elegans
MELSPNLKSNLNSEMNDLVQQAEEWAQSQHRSASPGALKDPKLCAIYKMILQNSALLLIEKSGVKTVEEFQKVFGPIIEEQKNKEDEIIEFEEFEEIEKARAELASPTSFTKYSMMLDEPLSASPFNRRRTSLFSDLHVLSPIASASSESFEKPKRFASSTQHAKKSFVTVESTRTPWRRSNSMYLEDVLNRTINTLLPPTEVPQCFKTESFDTITSSWRNAIGESSE

SEQ ID NO: 43 - DNA, 2334 bp, C. elegans
  CDS (1)..(2331)
atgagcgaagaggattggccttcgccgaaatttcgagagcatgttattcagagacttgagcctgagttggcgagaaaccggcagaatgccccaaatttaccagtacccggtgacgcgaggcaggtcgaagaatatgtgttcgcaaagtgtatgtcgaaggacgaatatatgaggacgatcgcaaaagtgattaatgctatcaattgtaatagtaaatcagcggcagtgccttccgtgttacaaccatcacagtttcatagtccgccgtgtacgacagcaggaaacactccggctggcggaacacccggatatcgagctcctgtgcctccagatccacaaccaacatcagctcaggcaagaaatccacctgttaccgtagcaacaacacaggcatcaacaactccatcagcaccaaatccgccaggaggattgcctgctccatcagcttctgccactgctgctgttgccgccgcggttgcttcatttccatcaccagatacaagtattcgtccaggtggacagatcactccaggatcacaggcacccggtggaggtccgacgccggcaccaaatgttccattcccaaatggaagcagccagatgaatggtggaccagcaatgggtcagccaccaccgcaaatgggtgctccaaatatgggtggaccaccgaatggatacggtggttacggtatgatgaatggaccacctggtagtggtgctccaatgggtggaaatccgtacaatcagcagattaaaaaggatatggatcaagctcgtccttgggatccttcagcacatatgtatcaacaacaaccacaatggggtgcaatgccaccacaacaaccacatggatatcctggaagaccaatgaacggtcaaggagccactccaaccggtccttcatcagttcttgaatcacttatcaatcagcctcaacaatatccaggacatcacaatcagatgggcccacctggagatcgaaatgttgctgctcaacgtgcagctgcccaacaacagcaacaacaacaacaacagcagcaacagcaacgacccggaatggttccaaaccagggaatgatgagttccgaggatcaaaccgtgtattctgcgaaattgagaaatatgcgtggaagctgcgacagtttgagaacacgagcaaggcaatgcagacatgagggtaaccatgaagctgctcacaagcttgaggttatgctcagtgtgctcgaaggaaaacgtgtggttagccttgaatacttgaaccatttggagatgtggattgccagaaagcaagacttcttgaatattgctccaatgtcacagaatcagaatcatatgggaatgaacgatccgatgatgaacggtgaacatgctatgttgggtaatggtcaggtgccaaatccctacggaggacatccaggttatggacatcagcagtatatgggacctccaccaccacatatgcaaatgcatcaaccaccaatgtggcatcaacagcaacaccaacagcagcaacgaatgatgccacaggatcatatgatgatgcagggtggaggtggtccagttcatggaatgtatcgtggagatatgggacacgatccgatgacatcaccagtcaacaatcatcgtcatgctccatacccgaatcctgcagcgatgagaaacaatatgagaatgccaaatggtcctggaccaattggaagagatcgtaattcgatgagtggatcaagtatgtcaggtccaagttctggagccccttcaatgaatccaatgggtacaccgaatcaaaaaatgggaacacctggatcaatgggtggaatgtcaggacttgatgatctgaactatgatgatttccttccaaacccaacgccgattgatgcacttcagccaacattgcacgtcgggcagaattcgatgaatgctggtccaccagttcaacgatcaaatctcaatgaaacagcaagaaaggaacttcaaatccttgatgctcgattcgagattgatccaaaccatcaacgtcatgatgccaatcatatcattgtcgtctgtaaaatccgtaatcaacagttcccaccacttcgactcgttgttccaaccacttatccagctggaaatgtgactgtagatcgagcagtcattgatttggatgcatatttgtatgatgatctacaaaattcagtatatgagagattatcacgacccggtctttcgtcaattactgactatttgaatgcttgggaagaacaagtgaatcaataccaaaaccagacaagcggtggtctagacgtcgctttcaacgtgggcaacgatttcttctacgataatcttaacttgtaa

SEQ ID NO: 44 - protein, 777 aa, C. elegans
MSEEDWPSPKFREHVIQRLEPELARNRQNAPNLPVPGDARQVEEYVFAKCMSKDEYMRTIAKVINAINCNSKSAAVPSVLQPSQFHSPPCTTAGNTPAGGTPGYRAPVPPDPQPTSAQARNPPVTVATTQASTTPSAPNPPGGLPAPSASATAAVAAAVASFPSPDTSIRPGGQITPGSQAPGGGPTPAPNVPFPNGSSQMNGGPAMGQPPPQMGAPNMGGPPNGYGGYGMMNGPPGSGAPMGGNPYNQQIKKDMDQARPWDPSAHMYQQQPQWGAMPPQQPHGYPGRPMNGQGATPTGPSSVLESLINQPQQYPGHHNQMGPPGDRNVAAQRAAAQQQQQQQQQQQQQRPGMVPNQGMMSSEDQTVYSAKLRNMRGSCDSLRTRARQCRHEGNHEAAHKLEVMLSVLEGKRVVSLEYLNHLEMWIARKQDFLNIAPMSQNQNHMGMNDPMMNGEHAMLGNGQVPNPYGGHPGYGHQQYMGPPPPHMQMHQPPMWHQQQHQQQQRMMPQDHMMMQGGGGPVHGMYRGDMGHDPMTSPVNNHRHAPYPNPAAMRNNMRMPNGPGPIGRDRNSMSGSSMSGPSSGAPSMNPMGTPNQKMGTPGSMGGMSGLDDLNYDDFLPNPTPIDALQPTLHVGQNSMNAGPPVQRSNLNETARKELQILDARFEIDPNHQRHDANHIIVVCKIRNQQFPPLRLVVPTTYPAGNVTVDRAVIDLDAYLYDDLQNSVYERLSRPGLSSITDYLNAWEEQVNQYQNQTSGGLDVAFNVGNDFFYDNLNL

SEQ ID NO: 45 - DNA, 1164 bp, C. elegans
  CDS (1)..(1161)
atgctatcaccttccgatcaagaaattgtagcggaaaatgctcggcgagctcttgatttgctcgacgatcaggtcttcggactaacggaacaagagaggcaaatcgagaatattgtctatgattttctgcagtcgaaagatggaaaatgtgctcttcttgaaggagaacgaaattgcggccgagaaagcacacttcggaaggttctcaacaaatatcacaaggaattacgagtaatcaacgcaggcttcgttgccgccgatccaaattatcagcaggaactcgcaaaggaggatgacgaaggatattttgtgtttttagtgcgagacgccgacaaattaatcactccacagcagcaaaagttcctctacacgatggttgacaaggcgacacacttctcgtggctcgtctttttctccgtttcccgacacgatttcgctcagaaccttcagaaacaggcgacgtcgagaattccgaaaattcaggtgtcatttgacgcgacacacgattatgaggagtattgtgcagcgttgacgcagtttttgattcctggcggctcaaaatgcgaagaatccgagtgctataagagatttgtgaagaatttgaactttcagacatggcttaggccggtttttgagacaagtcactacgtcgttctgaaacaattggccgcgcaattcctgatgcttttcggttactattgtgacgtagaagacacgtggaaacccgaggaaatcagcaaacagattcgtaaaattgtcgagacgacttctccctcgtataatggaaaattgttgattttaaaagatcaaacagttcgagcacaatgcgtttttctttgtgtctggagacttgtgcagaataaagagtcagatggaccacttttggcgccaaaaacttcgtatcgaaacgtttttctagaattcaaaaaactcgttaatcaacactatcctccactgaacgtgagcagtgatgtctacgtattccgagaattggatcatctcgttaaaatgggaattttgaaggcagacgagagcacaaatgtgacaaatacctcgtttcgaaaagtttggcttcatatcaatgataaaattgtggaggactcgatctcgaagttgcagctcccgcgagttgtttccgacttttttatgactattttgaagtaa

SEQ ID NO: 46 - protein, 387 aa, C. elegans
MLSPSDQEIVAENARRALDLLDDQVFGLTEQERQIENIVYDFLQSKDGKCALLEGERNCGRESTLRKVLNKYHKELRVINAGFVAADPNYQQELAKEDDEGYFVFLVRDADKLITPQQQKFLYTMVDKATHFSWLVFFSVSRHDFAQNLQKQATSRIPKIQVSFDATHDYEEYCAALTQFLIPGGSKCEESECYKRFVKNLNFQTWLRPVFETSHYVVLKQLAAQFLMLFGYYCDVEDTWKPEEISKQIRKIVETTSPSYNGKLLILKDQTVRAQCVFLCVWRLVQNKESDGPLLAPKTSYRNVFLEFKKLVNQHYPPLNVSSDVYVFRELDHLVKMGILKADESTNVTNTSFRKVWLHINDKIVEDSISKLQLPRVVSDFFMTILK

SEQ ID NO: 47 - DNA, 3003 bp, C. elegans
  CDS (1)..(3000)
atgacaattaatataaaatactcttccaaattttcttcttcaaagacatccagttcagaagagctgaaacctaaaacatacattccagcttattaccagccaccagtttctatgcctaaatactacgttaactggttgagaataaagttatccttgaataaactcaaaaatataagggcaatatatttgtttgaccaatgccaaaactttaaccagtatcaggaaagtagccgaaagttttcagctggtcaagttagctcattaactccatggtactccaatttcagtaattattctacagtaatcctccggatgaaggacacgtctcttccgccacttgaaaacccatcaaacgggggaacatatctctttaacctaattaccgtattcccctccattgcgctttcattcaattattcgtggcggggggacactgggccgtccatctcgattttctctttctctctctctgttctttttttttctcttttccctcaacataaaaatatatgcgcgcgcgcctggtgccgtcctttccgccgctctctttctttttctcttcgtttcataatgtcgagtgaacctgcgtcttcgtctacagaaaaagtcccggaggagccacatccacactctatcaagcacaaatttcaagggccacaatttgtgatcccccgagcattgagcgattacgtgttgaatgtgaacaatcagactcctgaatcttatgaaaaagcactgaacgccaaatatggaagagatgactacatcaccttatgcctccacgtcacaagtctttgcacagaatatggcccacttgatgacgggcctgaatatgtacttctttgtgattcacgagctcgtgttgacaagctgatcgaggatctcgtcaagttgttagagatcgacacagactacgtacaactagagttgcatggtggaaagcgccttcatcttcagaaacccgatgcagttctccgtgatattgcatataaacagagcaacgagggatctgacaagttctttttggagatgaagcttgtgccttcggagtcaatgaaagcgaaaattatgaagcaagaggaagaagaagagaaagcaagaaaacatgggcaatatcaacaatatcaagagtatcaccaacaacatcaagcaatgaatgatggacaaagtagctcgagtgtaccttcaactagctctccgtcatgctccagtgaggcaaatcggaaggaaatggaaacagtgcgtgaaccagctggaccgtcagaattgatgagggctataaatgctccagttgctcctgccccggttgtaatcaagattgaaactcctgttgcattgccagaagaagatgaaacactgatggatgatgatgaaatgccttcgctaactgtagaagcaccaagtgaagaagcttcatttgaagctgaacaaccttctccacaagtcccacaagcctcaattgagggaccatctcaacagcaacaaattcctggaacttctcaacagaaaagacaagttgctcgtggatctcgtactaatatgatttcctatcatgatttaccacctggcactggaaatgctcctccgatggcttgtcctcaagttacactcaaacttgaaaaaaatgttccatttgaagcgaaaatccgtgcagttgctgggtacactcgaaaaccaatttctgaagttcaaaaaatgagaccgagtgatctggagagcatcttccattcgatctgcattgccagcgttcaaagaataaagagaagaaatgaacttgttcaacagctacaggaaatcaatgcccaatcttgtaaaagtccgacaatgactatgaacaagaagttcacattggcaaaggcgtatcaaagggtccagaatgaaattgaaaaaattgatcgagagcaaatacttcctcaacagtacatgaacatgccaccaatgccaccacaaggacagcaaagattgcctccaccagcatatccacctggtattctgccaccacaacagaatcgtcaacaaggcgtacctccacaattccaacgttcaccgcaatttatgattggaccagatggacagcgttatgcacatccatatatgcaacttccaaattcaaaccaaagagctcgaattctgaatacttcatctgtgcagccgtctgaagaagtacgcaatcgcctcgtgaagattgaagcaatggcgatgaatatggcacaactgaacccacctcgaccacctcctccacagccaccacatcgagctctccaaggagaactgcagtttttgagacctggagcaccggatccgtgcaactttaggccagattctaagcagacatataacaatacatatgtgactgttgcatcaccagcgacgctcaccaattcaatcattccatggcacttcccgccatatgaaaaatcaggaagattgaatgtgagcaacacaataaaagctatcaacgagtatcgtcttctgtgcaattctcgacaggctgatccagcaagtttcctcgaattctacttccttggtgatccaatgccacacttcaataaaattctttcaattgctgattacaatatgtatttgtctcgaagacgatgcgatgaggcagatgtcaagattcatcgaatgtcgcacagtgatcaattgcaattgtaccttttggaacttcaatccgacgagtcaaatgtggaaaaatggaagacattctatagaattatgcagtgggatttgccactaaacaatgagtttccaagaatacttctaccatcttcattagacatcggaagaccagttgttgatagaaagaagaaatcaattgatcaggttatgaatcatatacatcgaatgcatagtcaacgcccaccaagcatgggaaactcttccacatcttctgaagcttcatctaccagtccaacaaatgctgcaacagcgaccagttctccagcatcgaaccgaccaacgacttctacagcacaaccaccaactttaaatccaacataa

SEQ ID NO: 48 - protein, 1000 aa, C. elegans
MTINIKYSSKFSSSKTSSSEELKPKTYIPAYYQPPVSMPKYYVNWLRIKLSLNKLKNIRAIYLFDQCQNFNQYQESSRKFSAGQVSSLTPWYSNFSNYSTVILRMKDTSLPPLENPSNGGTYLFNLITVFPSIALSFNYSWRGDTGPSISIFSFSLSVLFFSLFPQHKNICARAWCRPFRRSLSFSLRFIMSSEPASSSTEKVPEEPHPHSIKHKFQGPQFVIPRALSDYVLNVNNQTPESYEKALNAKYGRDDYITLCLHVTSLCTEYGPLDDGPEYVLLCDSRARVDKLIEDLVKLLEIDTDYVQLELHGGKRLHLQKPDAVLRDIAYKQSNEGSDKFFLEMKLVPSESMKAKIMKQEEEEEKARKHGQYQQYQEYHQQHQAMNDGQSSSSVPSTSSPSCSSEANRKEMETVREPAGPSELMRAINAPVAPAPVVIKIETPVALPEEDETLMDDDEMPSLTVEAPSEEASFEAEQPSPQVPQASIEGPSQQQQIPGTSQQKRQVARGSRTNMISYHDLPPGTGNAPPMACPQVTLKLEKNVPFEAKIRAVAGYTRKPISEVQKMRPSDLESIFHSICIASVQRIKRRNELVQQLQEINAQSCKSPTMTMNKKFTLAKAYQRVQNEIEKIDREQILPQQYMNMPPMPPQGQQRLPPPAYPPGILPPQQNRQQGVPPQFQRSPQFMIGPDGQRYAHPYMQLPNSNQRARILNTSSVQPSEEVRNRLVKIEAMAMNMAQLNPPRPPPPQPPHRALQGELQFLRPGAPDPCNFRPDSKQTYNNTYVTVASPATLTNSIIPWHFPPYEKSGRLNVSNTIKAINEYRLLCNSRQADPASFLEFYFLGDPMPHFNKILSIADYNMYLSRRRCDEADVKIHRMSHSDQLQLYLLELQSDESNVEKWKTFYRIMQWDLPLNNEFPRILLPSSLDIGRPVVDRKKKSIDQVMNHIHRMHSQRPPSMGNSSTSSEASSTSPTNAATATSSPASNRPTTSTAQPPTLNPT

SEQ ID NO: 49 - DNA, 186 bp, C. elegans
  CDS (1)..(183)
atgctccaccaccaacacgctcacgtggactgtgatatttcaactggacgattattcatcaccgacttcaacaatcaccgactcctgtcgcctcgtcgcctcggaacgtgtcacctacatcgaccaacccgtccccatccgaataattttctattttttttcaaattttattttatttttttataa

SEQ ID NO: 50 - protein, 61 aa, C. elegans
MLHHQHAHVDCDISTGRLFITDFNNHRLLSPRRLGTCHLHRPTRPHPNNFLFFFKFYFIFL

(19) United States
(12) Patent Application Publication
(10) Pub. No.: US 2005/0101773 A1
(43) Pub. Date: May 12, 2005

(54) Genes required for viability and/or reproduction in c. elegans and their use in the development of anti-nematode agents

(76) Inventors: Christophe Echeverri, Dresden (DE); Pierre Goenczy, Lausanne (CN); Anthony Hyman, Dresden (DE); Steven Jones, Vancouver (CA); Karen Oegema, La Jolla, CA (US); Matthew Kirkham, Brisbane (AU)

Correspondence Address: FOLEY AND LARDNER;SUITE 500, 3000 K STREET NW, WASHINGTON, DC, 20007, US

(21) Appl. No.: 10/415,656

(22) PCT Filed: Nov. 9, 2001
(86) PCT No.: PCT/EP01/13038

Related U.S. Application Data
(60) Provisional application No. 60/246,721, filed on Nov. 9, 2000.

(51) Int. Cl. C07H 21/02; C07H 21/04

(52) U.S. Cl. 536/23.1

(57) ABSTRACT

The present invention relates to several C. elegans genes and gene products identified by means of RNA-mediated interference (RNAi) as required for the viability, growth or reproduction of nematodes and to functional orthologues of said genes and gene products found in other nematode species, including all biologically-active derivatives thereof. The invention also comprises the use of said genes and gene products in the development of anti-nematode or other pesticidal agents and in methods for diagnosis and treatment of diseases associated with the infection or presence of nematodes.

In a first aspect, the present invention relates to several C. elegans genes and their corresponding gene products identified by means of RNA-mediated interference (RNAi) as required for the viability, growth or reproduction of nematodes.

In a second aspect, the invention is related to functional orthologues of said genes and gene products found in other nematode species, including all biologically-active derivatives thereof.

In a third aspect, the invention also comprises the use of said genes and gene products in the development of anti-nematode or other pesticidal agents and in methods for diagnosis and treatment of diseases associated with the infection or presence of nematodes.

In a forth aspect, the invention relates to antibodies to said gene products and their use in the development of anti-nematode agents and in methods for diagnosis and treatment of diseases associated with the infection or presence of nematodes.

In a fifth aspect, the present invention comprises the use of said genes and gene products for developing structural models or other models for evaluating drug binding and efficacy as well as to any other uses which are derived from the new functions described here and which will become apparent from the disclosure of the present application for any person skilled in the art.

The present invention is based on the silencing of the expression of specific genes by means of RNA-mediated interference (RNAi) to identify genes that are required for the normal growth, viability, or reproduction of the nematode worm C. elegans. As will be described later, there are many species of nematode that are parasitic to plants and animals including humans.

Therefore the task of the present invention is to identify new potential target genes/proteins that are required for the normal growth, viability or reproduction of nematodes to control nematode infection or infestation. These target genes/proteins can be used to screen for drugs that inhibit or effect the gene product and thus have the potential to be useful for the control of harmful or disease-causing nematodes. Particularly useful are those genes that exhibit little or no sequence conservation outside of nematodes. Indeed, drugs that specifically inhibit the products of such genes will be less likely to effect other species, including the human, animal, or plant that is host to the parasitic nematode.

The present invention solves this task by using RNAi to screen for genes that are required for the normal growth, viability, or reproduction of the nematode worm C. elegans. Furthermore, the invention solves this task by providing a number of new candidate target genes that have been validated as useful for the discovery of new approaches for the control of nematode diseases, infections or infestations.

Recently a new technique called RNA-mediated interference (RNAi) has been developed for determining metazoan gene functions (Fire, A. et al. Potent and specific genetic interference by double-stranded RNA in Caenorhabditis elegans. Nature 391, 806-811 (1998)). This technique consists in the targeted, sequence-specific inhibition of gene expression, as mediated by the introduction into an adult worm of double-stranded RNA (dsRNA) molecules corresponding to portions of the coding sequences of interest. By combining this method with the available genome sequences, and the use of a simple assay for determining nematode viability and fertility, a large number of genes can thus be functionally analyzed with unprecedented speed and efficiency (Gönczy, P. et al. Dissection of cell division processes in the one cell stage Caenorhabditis elegans embryo by mutational analysis. J Cell Biol 144, 927-946 (1999); Sulston, J. E., Schierenberg, E., White, J. G. & Thomson, J. N. The embryonic cell lineage of the nematode Caenorhabditis elegans. Dev. Biol. 100, 64-119 (1983)).

Therefore a large-scale RNAi technique-based screen was performed for 2,232 (that means 96%) of the predicted open reading frames on chromosome III of C. elegans which is described in detail in Gönczy et al., “Functional genomic analysis of cell division in C. elegans using RNAi of genes on chromosome III” Nature 408, 331-336 (2000). For the performance of this large scale screen double-stranded RNA corresponding to the individual open reading frames was produced and micro-injected into adult C. elegans hermaphrodites. Two days later it was checked with a stereomicroscope whether F1 larvae are present, as well as their developmental stage. The C. elegans genes provided by SEQ ID NO. 1-50 gave rise to a phenotype implying-a functional role of these-genes in nematode viability, growth and/or reproduction.

Table 1 provides a listing of the specific C. elegans genes identified and of their corresponding accession numbers in the Genbank/EMBL data bank from which the sequences of SEQ ID NO. 1 to 50 are derived (The C. elegans Sequencing Consortium. Genome sequence of the nematode C. elegans: a platform for investigating biology. Science 282, 2012-2018 (1998)). For all genes listed here, BLAST analysis of cross-species sequence similarities with all available online sequence databases indicate no significant homologies outside of nematodes.

Table 2 provides a listing of the specific primers used to generate the dsRNAs which were designed and used to specifically silence the expression of the C. elegans target genes.

In a first aspect, the present invention relates to an isolated nucleic acid molecule encoding a polypeptide required for the viability and/or growth and/or reproduction of nematodes or a fragment thereof and comprising a nucleic acid sequence selected from the group consisting of:
a) the nucleic acid sequences presented in SEQ ID NO. 1, 3, 5, 7, 9, 11, 14, 16, 18, 20, 22, 24, 26, 27, 29, 31, 33, 35, 37, 39, 41, 43, 45, 47 or 49 and fragments thereof and their complementry strands,
b) nucleic acid sequences encoding polypeptides that exhibit a sequence identity with any sequence provided by SEQ ID NO. 2, 4, 6, 8, 10, 12, 13, 15, 17, 19, 21, 23, 25, 30, 32, 34, 36, 38, 40, 42, 44, 46, 48 or 50 of at least 25% over 100 residues and/or that are detectable in a computer-aided search using the blast sequence analysis programs with an e-value of 10⁻³⁰,
c) nucleic acids which are capable of hybridising with the nucleic acid sequences of a) or b) under conditions of medium stringency,
d) nucleic acids which are degenerate as a result of the genetic code to any of the sequences-listed in a), b) or c).
   a):

The isolated nucleic acid molecules encoding a polypeptide required for the viability and/or growth and/or reproduction of nematodes or a fragment thereof as mentioned in a) are provided by the SEQ ID NO. 1, 3, 5, 7, 9, 11, 14, 16, 18, 20, 22, 24, 26, 27, 29, 31, 33, 35, 37, 39, 41, 43, 45, 47 or 49 and fragments thereof.

Their deduced amino acid sequences are provided by SEQ ID NO. 2, 4, 6, 8, 10, 12, 13, 15, 17, 19, 21, 23, 25, 30, 32, 34, 36, 38, 40, 42, 44, 46, 48 and 50.

b):

Additionally, the present invention also comprises isolated nucleic acid molecules that are structurally and functionally homologous counterparts (particularly orthologues) of at least one of said target genes as disclosed in SEQ ID NO. 1, 3, 5, 7, 9, 11, 14, 16, 18, 20, 22, 24, 26, 27, 29, 31, 33, 35, 37, 39, 41, 43, 45, 47 or 49.

Those homologous nucleic acid molecules may encode polypeptides that exhibit a sequence identity with any sequence provided by SEQ ID NO. 2, 4, 6, 8, 10, 12, 13, 15, 17, 19, 21, 23, 25, 30, 32, 34, 36, 38, 40, 42, 44, 46, 48 or 50 of at least 25% over 100 residues, preferably of at least 30% over 100 residues, more preferably of at least 35% over 100 residues and most preferably of at least 40% over 100 residues.

The invention also comprises isolated nucleic acid molecules that are detectable in a computer aided search using one of the BLAST sequence analysis programs with an e-value of at most 10^{-30,} preferably with an e-value of at most most 10^{-35,} more preferably with an e-value of at most most 10⁻⁴⁰.

The BLAST sequence analysis programs are programs used for sequence analysis that are publically available and known to anyone skilled in the art. When sequence alignments are done by a BLAST sequence analysis program, most of those programs calculate so called “e-values” to characterize the grade of homology between the compared sequences. Generally a small e-value characterizes a high sequence identity/homology, whereas larger e-values characterize lower sequence identities/homologies.

“Homology” means the degree of identity between two known sequences. As stated above, homologies, that means sequence identities, may suitably be determined by means of computer programs known in the art. The degree of homology required for the sequence variant will depend upon the intended use of the sequence. It is well within the capability of a person skilled in the art to effect mutational, insertional and deletional mutations which are designed to improve the function of the sequence or otherwise provide a methodological advantage.

c):

The present invention further relates to isolated nucleic acid sequences or fragments thereof which are capable of hybridizing with the nucleic acid sequences of (a) or (b) under conditions of medium/high stringency.

The grade of sequence identity between a first and a second nucleic acid molecule can also be characterized by the capability of the first nucleic acid molecule to hybridize under certain conditions to a second nucleic acid molecule.

Suitable experimental conditions for determining whether a given DNA or RNA sequence “hybridizes” to a specified polynucleotide or oligonucleotide probe involve presoaking of the filter containing the DNA or RNA to examine for hybridization in 5×SSC (sodium chloride/sodium citrate) buffer for 10 minutes, and prehybridization of the filter in a solution of 5×SSC, 5× Denhardt's solution, 0.5% SDS and 100 mg/ml of denaturated sonicated salmon sperm DNA (Maniatis et al., 1989), followed by hybridization in the same solution containing a concentration of 10 ng/ml of a random primed (Feinberg, A. P. and Vogelstein, B. (1983), Anal. Biochem. 132:6-13), ³²P-dCTP-labeled (specific activity >1×10⁹ cpm/μg) probe for 12 hours at approximately 45° C. The filter is then washed twice for 30 minutes in 2×SSC, 0.5% SDS at at least 55° C. (low stringency), at least 60° C. (medium stringency), preferably at least 65° C. (medium/high stringency), more preferably at least 70° C. (high stringency) or most preferably at least 75° C. (very high stringency). Molecules to which the probe hybridizes under the chosen conditions are detected using an x-ray film.

d):

The present invention further relates to isolated nucleic acid molecules or fragments thereof which are degenerate as a result of the genetic code to any of the sequences defined in (a), (b) or (c).

The application of automated gene synthesis provides an opportunity for generating sequence variants of the naturally occurring genes. It will be appreciated, for example, that polynucleotides coding for the same gene products can be generated by substituting synonymous codons for those represented in the naturally occurring polynucleotide sequences as identified herein. Such sequences will be referred to as “degenerate” to the naturally occurring sequences. In addition, polynucleotides coding for synthetic variants of the corresponding amino acid sequences can be generated which, for example, will result in one or more amino acids substitutions, deletions or additions. Also, nucleic acid molecules comprising one or more synthetic nucleotide derivatives (including morpholinos) which provide said nucleotide sequence with a desired feature, e.g. a reactive or detectable group, can be prepared. Synthetic derivatives with desirable properties may also be included in the corresponding polypeptides. All such derivatives and fragments of the above identified genes and gene products showing at least part of the biological activity of the naturally occurring sequences or which are still suitable to be used, for example; as probes for, e.g. identification of homologous genes or gene products, are included within the scope of the present invention.

Having herein provided the nucleotide sequences of various genes required for the viability and/or growth and/or reproduction of nematodes, it will be appreciated that automated techniques of gene synthesis and/or amplification may be used to isolate said nucleic acid molecules in vitro. Because of the length of some coding sequences, application of automated synthesis may require staged gene construction, in which regions of the gene up to about 300 nucleotides in length are synthesized individually and then ligated in correct succession for final assembly. Individually sythesized gene regions can be amplified prior to assembly, using polymerase chain reaction (PCR) technology. The technique of PCR amplification may also be used to directly generate all or part of the final genes/nucleic acid molecules. In this case, primers are synthesized which will be able to prime the PCR amplification of the final product, either in one piece or in several pieces that may be ligated together. For this purpose, either cDNA or genomic DNA may be used as the template for the PCR amplification. The cDNA template may be derived from commercially available or self-constructed cDNA libraries.

In a second aspect, the invention relates to nucleic acid probes comprising a nucleic acid sequence as previously characterized under (a) to (d) which may be a polynucleotide or an oligonucleotide comprising at least 15 nucleotides containing a detectable label. These nucleic acid probes may be synthesized by use of DNA synthesizers according to standard procedures or, preferably for long sequences, by use of PCR technology with a selected template sequence and selected primers. In the use of the nucleotide sequences as probes, the particular probe may be labeled with any suitable label known to those-skilled in the art, including radioactive and non-radioactive labels. Typical radioactive labels include ³²P, ¹²⁵I, ³⁵S, or the like. A probe labeled with a radioactive isotope can be constructed from a DNA template by a conventional nick translation reaction using a DNase and DNA polymerase. Non-radioactive labels include, for example, ligands such, as biotin or thyroxin, or various luminescent or fluorescent compounds. The probe may also be labeled at both ends with different types of labels, for example with an isotopic label at one end and a biotin label at the other end. The labeled probe and sample can then be combined in a hybridization buffer solution and held at an appropriate temperature until annealing occurs.

The invention also includes an assay kit comprising either an isolated nucleic acid molecule as defined above or a fragment thereof or a probe as defined above in a suitable container.

Duplex formation and stability depend on substantial complementarity between the two strands of a hybrid and a certain degree of mismatch can be tolerated. Therefore, the nucleic acid molecules and probes of the present invention may include mutations (both single and multiple), deletions, insertions of the above identified sequences, and combinations thereof, as long as said sequence variants still have substantial sequence homology to the original sequence which permits the formation of stable hybrids with the target nucleotide sequence of interest.

The above identified nucleic acid sequences and probes coding for polypeptides required for the viability and/or growth and/or reproduction of nematodes or a part thereof will have a wide range of useful applications, including their use for identifying homologous, in particular orthologous, genes in the same or different species, their use in screening assays for interacting drugs that inhibit, stimulate or effect worm growth, viability or reproduction, their use for developing computational models, structural models or other models for evaluating drug binding and efficacy, and their use in a method for diagnosis or treatment of nematode-associated diseases, in particular calabar swellings, lymphatic filariasis (elephantiasis) or onchocercoma.

Examples of nematodes associated with human or animal diseases include, but are not limited to, Wuchereria bancrofti, Brugia malaya, Loa loa and Onchocerca volvulus. Examples of nematodes associated with plant diseases include, but are not limited to, Heterodera, including H. glycines, H. avenae, H. schachtii, H. trifolii, H. gottingiana, H. cajani, H. zeae; Globodera, including G. rostochiensis, G. pallida, G. tabacum, Meloidogyne, including M. arenaria, M. incognita, M. javanica, M. hapla, M. chitwoodi; Ditylenchus, including D. destructor, D. dipsaci, D. angustus; Anguina, including A. tritici, A. agrostis, Afrina/Anguina wevelli; Pratylenchus, including P. penetrans, P. brachyurus, P. coffeae, P. zeae, P. goodeyi, P. thornei, P. vulnus; Radopholus, including R. similis; Hirschmanniella, including H. oryzae, H. mucronata, H. spinicauda; Hoplolaimus, including H. columbus, H. seinhorsti, H. indicus; Rotylenchulus, including R. reniformis; Tylenchulus, including T. semipenetrans; Helicotylenchus, including H. multicinctus, H. mucronatus, H. dihystera, H. pseudorobustus; Criconemella, including C. xenoplax, C. axestis, C. spharocephalum; Xiphinema, including X. americanum, X. elongatum; Longidorus, including L. africanus; Trichodorus; Paratrichodorus, including P. minor; Aphelenchs, including A. fragariae, A. besseyi, A. ritzemabosi, Bursaphelenchus xylophilus.

In a third aspect, the present invention relates to the use of the above identified nucleic acid molecules and probes for diagnostic purposes. This diagnostic use of the above identified nucleic acid molecules and probes may include, but is not limited to the quantitative detection of the expression of said target genes in biological probes (preferably, but not limited to cell extracts, body fluids, etc.), particularly by quantitative hybridization to the endogenous nucleic acid molecules comprising the above-characterized nucleic acid sequences (particularly cDNA, RNA). A nematode infection or a disease associated with the presence of nematodes as previously defined may be diagnosed that way.

In a fourth aspect, the present invention relates to the use of the above identified nucleic acid molecules, probes or their corresponding polypeptides for therapeutical purposes.

This therapeutical use of the above identified nucleic acid molecules, probes or their corresponding polypeptides may include, but is not limited to the use of said nucleic acid molecules and their corresponding polypeptides for direct or indirect inhibition of the expression of said target genes and/or for inhibition of the function of said target genes. Particularly gene therapy vectors, e.g. viruses, or naked or encapsulated DNA or RNA (e.g. an antisense nucleotide sequence) with the above-identified sequences might be suitable for the introduction into the body of a subject suffering from a disease or from a disease associated with the presence of nematodes or caused by nematode infection.

A particularly preferred therapeutical use of the above identified nucleic acid molecules or probes relates to their use in a therapeutical application of the RNAi technique, particularly in humans or in human cells.

Double-stranded RNA oligonucleotides effect silencing of the expression of gene(s) which are highly homologous to either of the RNA strands in the duplex. Recent discoveries reveal that this effect, called RNA interference (RNAi), that had been originally discovered in C. elegans, can also be observed in cells, particularly in human cells. Therefore the invention further comprises the use of double-stranded RNA oligonucleotides with the above identified nucleotide sequences (as stated in a) to d)), preferably with a length of at least 15 nucleotides (nt), more preferably with a length of at least 20 nt, for therapeutical silencing of the expression of genes involved in nematode viability and/or reproduction in cells of other species, particularly in human cells. This therapeutical use particularly applies to cells of an individual that suffers from a disease associated with nematode presence or infection, in particular calabar swellings, lymphatic filariasis (elephantiasis) or onchocercoma.

In a fifth aspect, the invention further comprises a nucleic acid construct or a recombinant vector having incorporated the nucleic acid molecules as defined in (a) to (d) or a fragment thereof.

“Nucleic acid construct” is defined herein as any nucleic acid molecule, either single- or double-stranded, in which nucleic acid sequences are combined and juxtaposed in a manner which will not occur naturally. The vector may be any vector which can be conveniently subjected to recombinant DNA procedures. The choice of the vector will usually depend on the host cell into which it is to be introduced. The vector may be an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the protein or fragment of interest is operably linked to heterologous or homologous control sequences. The term “control sequences” is defined herein to include all components which are necessary or advantageous for expression of the coding nucleic acid sequence. Such control sequences include, but are not limited to, a promoter, a ribosome binding site, translation initiation and termination signals and, optionally, a repressor gene or various activator genes. Control sequences are referred to as “homologous” if they are naturally linked to the coding nucleic acid sequence of interest and referred to as “heterologous” if this is not the case. The term “operably linked” indicates that the sequences are arranged so that they function in concert for their intended purpose, i.e. expression of the desired protein.

The promoter may be any DNA sequence which shows transcriptional activity in the host cell of choice and may be derived from genes encoding proteins either homologous or heterologous to the host cell.

Examples of suitable promoters for directing the transcription in a bacterial host are, e.g., the phage Lambda P_{R} or P_{L} promoters, the lac, trp or tac promoters of E. coli, the promoter of the Bacillus subtilis alkaline protease gene or the Bacillus licheniformis alpha-amylase gene.

Examples of suitable promoters for directing the transcription in mammalian cells are, e.g., the SV40 promoter (Subramani et al., Mol. Cell. Biol. 1 (1981), 854-864), the MT-1 (metallothionein gene) promoter (Palmiter et al., Science 222 (1983), 809-814) or the adenovirus 2 major late promoter.

Examples of suitable promoters for use in insect cells are, e.g., the polyhedrin promoter (Vasuvedan et al., Febs. Lett 311, (1992), 7-11), the Autographa californica polyhedrosis basic protein promoter (EP 397 485), or the baculovirus immediate early gene 1 promoter (U.S. Pat. No. 5,155,037, U.S. Pat. No. 5,162,222).

Examples of suitable promoters for use in yeast cells include promoters from yeast glycolytic genes (Hitzeman et al., J. Biol. Chem. 255 (1980), 1203-12080; Alber and Kawasaki, J. Mol. Appl. Gen. 1 (1982), 419-434) and the ADH2Ac promoter (Russell et al., Nature 304 (1983), 652-654).

The coding sequence may, if necessary, be operably linked to a suitable terminator, such as the human growth hormone terminator (Palmiter et al., supra), or a polyadenylation sequence. Also, to permit secretion of the expressed protein, a signal sequence may precede the coding sequence.

Further, the vector may comprise a DNA sequence enabling the vector to replicate in the host cell in question. Examples of such sequences are the origins of replication of the plasmids pUC19, pACYC177, pUB110, pE194, pAMB1 and pIJ702. Another example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene coding for a product which complements a defect iii the host cell, such as the gene coding for dihydrofolate reductase (DHFR) or a gene which confers resistance to a drug, e.g. ampicillin, kanamycin, tetracyclin, choramphenicol, neomycin or hygromycin.

A number of vectors suitable for expression in prokaryotic or eukaryotic cells are known in the art and several of them are commercially available.

Some commercially available mammalian expression vectors which may be suitable include, but are not limited to, pMC1neo (Stratagene), pXT1 (Stratagene), pSG5 (Stratagene), pcDNAI (Invitrogen), EBO-pSV2-neo (ATCC 37593), pBPV-1 (8-2) (ATCC 37110), pSV2-dhfr (ATCC 37146).

In a sixth aspect, the invention comprises host cells into which the nucleic acid construct or the recombinant vector is introduced. These host cells may be prokaryotic or eukaryotic, including, but not limited to, bacteria, fungal cells, including yeast and filamentous fungi, mammalian cells, including, but not limited to, cell lines of human, bovine, porcine, monkey and rodent origin, and insect cells including, but not limited to, drosophila derived cell lines.

The selection of an appropriate host cell will be dependent on a number of factors recognized by the art. These include, e.g., compatibility with the chosen vector, toxicity of the (co)products, ease of recovery of the desired protein or polypeptide, expression characteristics, biosafety and costs.

Examples of suitable prokaryotic cells are gram positive bacteria such as Bacillus subtilis, Bacillus licheniformis, Bacillus brevis, Streptomyces lividans etc. or gram negative bacteria such as E. coli.

The yeast host cell may be selected from a species of Saccharomyces or Schizosaccharomyces, e.g. Saccharomyces cerevisiae. Useful filamentous fungi may be selected from a species of Aspergillus, e.g. Aspergillus oryzae or Aspergillus niger.

Cell lines derived from mammalian species which may be suitable and which are commercially available include, but are not limited to, COS-1 (ATCC CRL 1650) COS-7 (ATCC CRL 1651), CHO-K1 (ATCC CCL-61), 3T3 (ATCCL 92), NIH/3T3 (ATCC CRL 1658), HeLa (ATCCL 2), and MRC-5 (ATCC CCL 171).

The expression vector may be introduced into the host cells according to any one of a number of techniques including, but not limited to, transformation, transfection, protoplast fusion, and electroporation.

The recombinant host cells are then cultivated in a suitable nutrient medium under conditions permitting the expression of the protein of interest. The medium used to cultivate the cells may be any conventional medium suitable for growing the host cells, such as minimal or complex media containing appropriate supplements. Suitable media are available from commercial suppliers or may be prepared according to published recipes (e.g. in catalogues of the American Type Culture Collection).

Identification of the heterologous polypeptide expressing host cell clones may be done by several means, including, but not limited to, immunological reactivity with specific antibodies.

In a seventh aspect, the invention is related to a method for producing a polypeptide required for viability, growth and/or reproduction of nematode worms, particularly C. elegans and nematode worms parasitic to humans, livestock, or plants or a fragment thereof in a host cell comprising the steps
(i) transferring the expression vector with an operably linked nucleic acid molecule as previously defined in (a) to (d) into a suitable host ceH, and
(ii) cultivating the host cells of step (i) under conditions which wlllpermit the expression of said polypeptide or fragment thereof and
(iii) optionally, secretion of the expressed polypeptide into the culture medium.

In an eighth aspect, the invention comprises a polypeptide required for nematode viability, growth and/or reproduction or a fragment thereof comprising an amino acid sequence selected from the group consisting of:
(a) the amino acid sequences depicted in SEQ ID NO. 2, 4, 6, 8, 10, 12, 13, 15, 17, 19, 21, 23, 25, 30, 32, 34, 36, 38, 40, 42, 44, 46, 48 or 50 and fragments thereof,
(b) amino acid sequences which exhibit a sequence identity with the sequences of (a) of at least 25% over 100 residues, preferably of at least 30% over 100 residues, more preferably of at least 35% over 100 residues and most preferably of at least 40% over a 100 residues and/or which are detectable in a computer aided search using the BLAST sequence analysis programs with an e-value of at most 10⁻³⁰, preferably with an e-value of at most 10⁻³⁵ and most preferably with an e-value of at most 10⁻⁴⁰,
(c) amino acid sequences encoded by a nucleic acid molecule that is capable of hybridizing with the nucleic acid sequences of (a) or (b) or encoded by a nucleic acid molecule that is degenerate as a result of the genetic code to any of the sequences as defined in (a) or (b).

The heterologous polypeptide may also be a fusion polypeptide in which another polypeptide is fused at the N-terminus or the C-terminus of the polypeptide or fragment thereof. A fused polypeptide is produced by fusing a nucleic acid sequence (or a portion thereof) encoding another polypeptide to a nucleic acid sequence (or a portion thereof) of the present invention. Techniques for producing fusion polypeptides are known in the art and include ligating the coding sequences so that they are in frame and the expression of the fusion polypeptide is under control of the same promotor(s) and terminator.

Expression of the polypeptides of interest may also be performed using in vitro produced synthetic in RNA. Synthetic mRNA can be efficiently translated in various cell-free systems, including but not limited to wheat germ extracts and reticulocyte extracts, as well as efficiently translated in cell based systems including, but not limited to, microinjection into frog oocytes, preferably Xenopus oocytes.

In a ninth aspect, the invention involves antibodies against the above identified polypeptides and against immunogenic fragments thereof.

The term “antibody” as used herein includes both polyclonal and monoclonal antibodies, as well as fragments thereof, such as Fv, Fab and F(ab)₂ fragments that are capable of binding antigen or hapten. The antibodies of the present invention will have a wide range of useful applications, including their use for affinity purification of the corresponding immunogenic (poly)peptides, their use for the preparation of anti-idiotypic antibodies, as well as their use as specific binding agents in various assays, e.g. diagnostic or drug-screening assays, or in a method for treatment of diseases associated with nematodes as exemplified above. Specifically, said antibodies or suitable fragments thereof, particularly in humanized form, may be used as therapeutic agents in a method for treating nematode-associated diseases. Also, antibodies may be raised to the most characteristic parts of the above identified polypeptides and subsequently be used to identify structurally and/or functionally related polypeptides from other sources as well as mutations and derivatives of the above identified polypeptides.

To raise antibodies against the polypeptides of the present invention, there may be used as an immunogen either the intact polypeptide or an immunogenic fragment thereof, produced in a suitable host cell as described above or by standard peptide synthesis techniques.

Polyclonal antibodies are raised by immunizing animals, such as mice, rats, guinea pigs, rabbits, goats, sheep, horses etc., with an appropriate concentration of the polypeptide or peptide fragment of interest either with or without an immune adjuvant.

Acceptable immune adjuvants include, but are not limited to, Freund's complete adjuvant, Freund's incomplete adjuvant, alum-precipitate, water-in-oil-emulsion containing Corynebacterium parvum and tRNA.

In a typical immunization protocol each animal receives between about 0.1 μg and about 1000 μg of the immunogen at multiple sites either subcutaneously (SC), intraperitoneally (IP), intradermally or in any combination thereof in an initial immunization. The animals may or may not receive booster injections following the initial injection. Those animals receiving booster injections are generally given an equal amount of the immunogen in Freund's incomplete adjuvant by the same route at intervals of about three or four weeks until maximal titers are obtained. At about 7-14 days after each booster immunization or about weekly after a single immunization, the animals are bled, the serum collected, and aliquots are stored at about −20° C.

Monoclonal antibodies which are reactive with the polypeptide or peptide fragment of interest are prepared using basically the technique of Kohler and Milstein, Nature 256: 495-497 (1975). First, animals, e.g. Balb/c mice, are immunized using a protocol similar to that described above. Lymphocytes from antibody-positive animals, preferably splenocytes, are obtained by removing spleens from immunized animals by standard procedures known in the art. Hybridoma cells are produced by mixing the splenocytes with an appropriate fusion partner, preferably myeloma cells, under conditions which will allow the formation of stable hybridomas. Fusion partners may include, but are not limited to: mouse myelomas P3/NS1/Ag 4-1; MPC-11; S-194 and Sp 2/0. Fused hybridoma cells are selected by growth in a selection medium and are screened for antibody production. Positive hybridomas may-be grown and injected into, e.g., pristane-primed Balb/c mice for ascites production. Ascites fluid is collected about 1-2 weeks after cell transfer and the monoclonal antibodies are purified by techniques known in the art Alternatively, in vitro production of monoclonal antibodies (mAb) is possible by cultivating the hybridomas in a suitable medium, e.g. DMEM with fetal calf serum, and recovering the mAb by techniques known in the art.

Recovered antibody can then be coupled covalently to a detectable label, such as a radiolabel, enzyme label, luminescent label, fluorescent label or the like, using linker technology established for this purpose.

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay techniques. Similar assays may be used to detect the presence of the above identified polypeptides or fragments thereof in body fluids or tissue and cell extracts.

Assay kits for performing the various assays mentioned in the present application may comprise suitable isolated nucleic acid or amino acid sequences of the above identified genes or gene products, labelled or unlabelled, and/or specific ligands (e.g. antibodies) thereto and auxiliary reagents as appropriate and known in the art. The assays may be liquid phase assays as well as solid phase assays (i.e. with one or more reagents immobilized on a support).

Unless-otherwise specified, the manipulations of nucleic acids and polypeptides/-proteins can be performed using standard methods of molecular biology and immunolog (see, e.g. Maniatis et al. (1989), Molecular cloning: A laboratory manual, Cold Spring Harbor Lab., Cold Spring Harbor, N.Y.; Ausubel, F. M. et al. (eds.) “Current protocols in Molecular Biology”. John Wiley and Sons, 1995; Tijssen, P., Practice and Theory of Enzyme Immunoassays, Elsevier Press, Amsterdam, Oxford, New York, 1985).

The invention further includes an assay kit comprising either the polypeptide as defined above or a fragment thereof or an antibody against said polypeptides as defined-above or against immunogenic fragments thereof.

The recombinant-polypeptides and fragments thereof, as well as antibodies against those polypeptides or immunogenic fragments thereof will have a wide range of useful applications, including their use in screening assays for interacting drugs that inhibit, stimulate or effect the growth, viability or reproduction of nematodes, their use for developing computational models, structural models or other models for evaluating drug binding and efficacy, and their use in a method for diagnosis or treatment of nematode-associated diseases, in particular calabar swellings, lymphatic filariasis (elephantiasis) or onchocercoma.

Therefore in a tenth aspect, the present invention explicitly includes the use of polypeptides as defined above or fragments thereof or of antibodies against said polypeptides or immunogenic fragments thereof in a screening assay for interacting drugs that inhibit, stimulate or effect nematode viability, growth and/or reproduction.

Such a screening assay for interacting drugs may particularly comprise, but is not limited to the following steps:
1. recombinant expression of said polypeptide or of an appropriate derivative thereof
2. isolation and optionally purification of the recombinantly expressed polypeptide or of its derivative, in particular by affinity chromatography
3. optionally labelling of the chemical compounds that are tested to interact with said polypeptide or its derivative and/or labelling of the recombinantly expressed polypeptide
4. immobilization of the recombinantly expressed polypeptide or of its derivative to a solid phase
5. binding of a potential interaction partner or a variety thereof to the immobilized polypeptide or its derivative
6. optionally one or more washing steps
7. detection and/or quantification of the interaction, in particular by monitoring the amount of label remaining associated with the solid phase over background levels.

Step 1 includes the recombinant expression of the above identified polypeptide or of its derivative from a suitable expression system, in particular from cell-free translation, bacterial expression, or baculusvirus-based expression in insect cells.

Step 2 comprises the isolation and optionally the subsequent purification of said recombinantly expressed polypeptides with appropriate biochemical techniques that are familiar to a person skilled in the art.

Alternatively, these screening assays may also include the expression of derivatives of the above identified polypeptides which comprises the expression of said polypeptides as a fusion protein or as a modified protein, in particular as a GST-fusion protein or as a protein bearing a so called “tag”-sequence. These “tags”-sequences consist of short nucleotide sequences that are ligated ‘in frame’ either to the N- or to the C-terminal end of the coding region of said target gene. One of the most common “tags” that are used to label recombinantly expressed genes is the “poly-Histidine-tag” which encodes a homopolypeptide consisting merely of histidines. In this context the term “polypeptide” does not merely comprise polypeptides with an amino acid sequences selected from the group of SEQ ID NO. 2, 4, 6, 8, 10, 12, 13, 15, 17, 19, 21, 23, 25, 30, 32, 34, 36, 38, 40, 42, 44, 46, 48 or 50, but also their naturally occuring homologues, preferably orthologues, and derivatives of these polypeptides, in particular fusion proteins or polypeptides comprising a “tag”-sequence.

These polypeptides, particularly those labelled by an appropriate tag-sequence (for instance a His-tag) or by GST, may be purified by standard affinity chromatography protocols, in particular by using chromatography resins linked to anti-His-tag-antibodies or to anti-GST-antibodies which are both commercially available. Alternatively to the use of anti-tag- or anti-GST-antibodies or other ‘label-specific’ antibodies the purification may also involve the use of antibodies against said polypeptides. Screening assays that involve a purification step of the recombinantly expressed target genes as described above (step 2) are preferred embodiments of this aspect of the invention.

In a third—optional—step the compounds tested for interaction may be labelled by incorporation of radioactive isotopes or by reaction with luminescent or fluorescent compounds. Alternatively or additionally also the recombinantly expressed polypeptide may be labelled.

In a forth step the recombinantly expressed polypeptide is immobilized to a solid phase, particularly (but not limited) to a chromatography resin. The coupling to the solid phase is thereby preferably established by the generation of covalent bonds.

In a fifth step a candidate chemical compound that might be a potential interaction partner of the said recombinant polypeptide or a complex variety thereof (particularly a drug library) is brought into contact with the immobilized polypeptide.

In a sixth—optional—step one or several washing steps may be performed. As a result just compounds that strongly interact with the immobilized polypeptide remain bound to the solid (immobilized) phase.

In step 7 the interaction between the polypeptide and the specific compound is detected, in particular by monitoring the amount of label remaining associated with the solid phase over background levels.

Description of the Sequence Protocol

SEQ ID NO. 1 shows the spliced DNA sequence of the C. elegans gene D2045.1 (3081 bp).
SEQ ID NO. 2 shows the deduced amino acid sequence of the C. elegans gene D2045.1 (1026 aa).
SEQ ID NO. 3 shows the spliced DNA sequence of the C. elegans gene C39B5.2. (1230 bp).
SEQ ID NO. 4 shows the deduced amino acid sequence of the C. elegans gene C39B5.2 (409 aa).
SEQ ID NO. 5 shows the spliced DNA sequence of the C. elegans gene C29E4.2 (2754 bp).
SEQ ID NO. 6 shows the deduced amino acid sequence of the C. elegans gene C29E4.2 (918 aa).
SEQ ID NO. 7 shows the spliced DNA sequence of the C. elegans gene H04J21.3 (2169 bp).
SEQ ID NO. 8 shows the deduced amino acid sequence of the C. elegans gene H04J21.3 (722 aa).
SEQ ID NO. 9 shows the spliced DNA sequence of the C. elegans gene C38C10.4 (1578 bp).
SEQ ID NO. 10 shows the deduced amino acid sequence of the C. elegans gene C38C10.4 (525 aa).
SEQ ID NO. 11 shows the spliced DNA sequence of the C. elegans gene F22B7.13 (1578 bp).
SEQ ID NO. 12 shows the deduced amino acid sequence of the C. elegans gene F22B7.13 (525 aa).
SEQ ID NO. 13 shows the deduced amino acid sequence of the C. elegans gene C239G10.9 (179 aa).
SEQ ID NO. 14 shows the spliced DNA sequence of the C. elegans gene F54C4.3 (3438 bp).
SEQ ID NO. 15 shows the deduced amino acid sequence of the C. elegans gene F54C4.3 (1145 aa).
SEQ ID NO. 16 shows the spliced DNA sequence of the C. elegans gene K12H4.5 (294 bp).
SEQ ID NO. 17 shows the deduced amino acid sequence of the C. elegans gene K12H4.5 (97 aa).
SEQ ID NO. 18 shows the spliced DNA sequence of the C. elegans gene R13F6.1 (450 bp).
SEQ ID NO. 19 shows the deduced amino acid sequence of the C. elegans gene R13F6.1 (149 aa).
SEQ ID NO. 20, 22, 24 shows the spliced DNA sequences of three C. elegans gene WO7B3.2 isoforms (1638 bp, 1707 bp and 1620 bp, respectively).
SEQ ID NO. 21, 23, 25 shows the deduced amino acid sequences of the three C. elegans gene WO7B3.2 isoforms (545 aa, 568 aa and 539 aa, respectively).
SEQ ID NO. 26 shows the spliced DNA sequence of the C. elegans gene Y66A7A.8 (1524 bp).
SEQ ID NO. 27 shows the spliced DNA sequence of the C. elegans gene Y49E10.22 (702 bp).
SEQ ID NO. 28 shows the deduced amino acid sequence of the C. elegans gene Y49E10.22 (233 aa).
SEQ ID NO. 29 shows the spliced DNA sequence of the C. elegans gene Y39E4B. 1 (738 bp).
SEQ ID NO. 30 shows the deduced amino acid sequence of the C. elegans gene Y39E4B.11 (245 aa).
SEQ ID NO. 31 shows the spliced DNA sequence of the C. elegans gene R02F2.7 (1812 bp).
SEQ ID NO. 32 shows the deduced amino acid sequence of the C. elegans gene R02F2.7 (603 aa).
SEQ ID NO. 33 shows the spliced DNA sequence of the C. elegans gene C45G9.5 (951 bp).
SEQ ID NO. 34 shows the deduced amino acid sequence of the C. elegans gene C45G9.5 (316 aa).
SEQ ID NO. 35 shows the spliced DNA sequence of the C. elegans gene F23H11.5 (291 bp).
SEQ ID NO. 36 shows the deduced amino acid sequence of the C. elegans gene F23H11.5 (96 aa).
SEQ ID NO. 37 shows the spliced DNA sequence of the C. elegans gene C32A3.2 (1041 bp).
SEQ ID NO. 38 shows the deduced amino acid sequence of the C. elegans gene C32A3.2 (346 aa).
SEQ ID NO. 39 shows the spliced DNA sequence of the C. elegans gene K04G7.1 (1677 bp).
SEQ ID NO. 40 shows the deduced amino acid sequence of the C. elegans gene K04G7.1 (558 aa).
SEQ II) NO. 41 shows the spliced DNA sequence of the C. elegans gene K11H3.2 (687 bp).
SEQ ID NO. 42 shows the deduced amino acid sequence of the C. elegans gene K11H3.2 (228 aa).
SEQ ID NO. 43 shows the spliced DNA sequence of the C. elegans gene R12B2.5 (2334 bp).
SEQ ID NO. 44 shows the deduced amino acid sequence of the C. elegans gene R12B2.5 (777 aa).
SEQ ID NO. 45 shows the spliced DNA sequence of the C. elegans gene Y39A1A.13 (1164 bp).
SEQ ID NO. 46 shows the deduced amino acid sequence of the C. elegans gene Y39A1A.13 (387 aa).
SEQ ID NO. 47 shows the spliced DNA sequence of the C. elegans gene ZK1236.3 (3003 bp).
SEQ ID NO. 48 shows the deduced amino acid sequence of the C. elegans gene ZK1236.3 (1000 aa).
SEQ ID NO. 49 shows the spliced DNA sequence of the C. elegans gene F23F12.2 (186 bp).

SEQ ID NO. 50 shows the deduced amino acid sequence of the C. elegans gene F23F12.2 (61 aa).

The following examples illustrate the present invention without, however, limiting the same thereto.

### EXAMPLE 1

### RNAi Experiments

First, oligonucleotide primer pair sequences were selected to amplify portions of the gene of interest's coding region using standard PCR-techniques. Primer pairs were chosen to yield PCR products containing at least 500 bases of coding sequence, or a maximum of coding bases for genes smaller than 500 bases. In order to permit the subsequent use of the PCR product as a template for in vitro RNA transcription reactions from both DNA strands, the T7 polymerase promoter sequence “TAATACGACTCACTATAGG” was added to the 5′ end of forward primers, and the T3 polymerase promoter sequence “AATTAACCCTCACTAAAGG” was added to the 5′ end of reverse primers. The synthesis of oligonucleotide primers was completed by a commercial supplier (Sigma-Genosys, UK or MWG-Biotech, Germany).

PCR reactions were performed in a volume of 50 μl, with Taq polymerase using 0.8 μM primers and approximately 0.1 μg of wild-type (N2 strain) genomic DNA template. The PCR products were EtOH precipitated, washed with 70% EtOH and resuspended in 7.0 μl TE. 1.0 μl of the PCR reaction was pipetted into each of two fresh tubes for 5 μl transcription reactions using T3 and T7 RNA polymerases. The separate T3 and T7 transcription reactions were performed according to the manufacturer's instructions (Ambion, Megascript kit), each diluted to 50 μl with RNase-free water and then combined. The mixed RNA was purified using RNeasy kits according to the manufacturer's instructions (Qiagen), and eluted into a total of 130 μl of RNase-free H₂O. 50 μl of this was mixed with 10 μl 6× injection buffer (40 mM KPO₄ pH 7.5, 6 mM potassium citrate, pH 7.5, 4% PEG 6000). The RNA was annealed by heating at 68° C. for 10 min, and at 37° C. for 30 min. Concentration of the final dsRNAs were measured to be in the range of 0.1-0.3 μg/μl. The products of the PCR reaction, of the T3 and T7 transcription reactions, as well as the dsRNA species were run on 1% agarose gels to be examined for quality control purposes. Success of double stranding was assessed by scoring shift in gel mobility with respect to single stranded RNA, when run on non-denaturing gels.

Double-stranded RNAs were injected bilaterally into the syncitial portion of both gonads of wild-type (N2 strain) young adult hermaphrodites, and the animals incubated at 20° C. for 24 hrs.

Three injected animals were then transferred to a fresh plate 24 hrs after injection of dsRNA, and left at 20° C. Two days later, the plate was checked with a stereomicroscope (20-40× total magnification) for the presence of F1 larvae (L2's-L4's), as well as their developmental stage. Two days after that, the plate was inspected again for the presence of F1 adults, as well as their overall body morphology and the presence of F2 progeny.

### EXAMPLE 2

### Protocol for Identifying Functional Orthologues in Other Species

The present invention describes genes identified as having essential functions for the viability and/or growth and/or reproduction of the model organism C. elegans. The basis for performing research in model organisms is that the newly discovered functions for the genes in C. elegans will be conserved in other nematode species. Many basic cell functions are highly conserved during evolution and therefore the approach of discovering a gene function in C. elegans and using the information to characterise or assign functions for orthologous genes in other nematode species is well justified. There are two themes of conservation of genes during evolution. A gene sequence may be conserved. This means that the DNA nucleotide sequence of the gene is very similar in different species, which in turn suggests that the function of the gene is the same in the different species. As is known to any person skilled in the art, a sequence identity or homology above a particular level defines that two genes in different species code for the same gene product and gene function. Homologous genes are typically identified by performing blast analysis with appropriate software, or by other approaches. For a blast search, an e-value of 10⁻³ will extract the significant homologous sequences. Further phylogenetic analysis can be performed to identify which of the extracted sequences are the orthologues.

Therefore the following example for identification of orthologues can be presented. A blast search is performed using the blast sequence analysis-programs and an e-value of 10⁻³. An alternative parameter can be the percentage of sequence identity. Over 100 residues, a sequence identity of 30% defines a homologous gene. After the blast search is completed, multiple sequence alignment is performed using appropriate software (for example, CLUSTALW) and a neighbour joining phylogenetic tree is generated. Any person skilled in the art can identify an orthologue from a phylogenetic tree. Essentially, a sequence that is separated on the tree by a single speciation event or most closely related on the tree is likely to be an orthologue.

The second theme of conservation is that the gene function can be conserved with greater divergence of sequence. In the present invention this theme of conservation is not defined. However, if other genes are discovered to have functions that result in the gene product being identified as the same gene product as those claimed in the present invention then the present claims also apply to such genes.

TABLE 1
Accession numbers and RNAi-induced Phenotypes

| | GenBank/ | |
|---|---|---|
| Gene Name | EMBL ID | Phenotype Description |
| D2045.1 | Z35639 | All F1 worms dead as unhatched embryos. |
| C39B5.2 | AC006617 | All F1 worms dead as unhatched embryos. |
| C29E4.2 | L23651 | All F1 worms dead as unhatched embryos. |
| H04J21.3 | AF040644 | All F1 worms dead as unhatched embryos. |
| C38C10.4 | Z19153 | All F1 worms dead as unhatched embryos. |
| F22B7.13 | L12018 | All F1 worms dead as unhatched embryos. |
| C23G10.9 | U39851 | All F1 worms dead as unhatched embryos. |
| F54C4.3 | AF099916 | All F1 worms dead as unhatched embryos. |
| K12H4.5 | L14331 | All F1 worms dead as unhatched embryos. |
| R13F6.1 | U00046 | All F1 worms dead as unhatched embryos. |
| W07B3.2 | AF100304 | All F1 worms dead as unhatched embryos. |
| Y66A7A.8 | (GenPEP ID) | All F1 worms dead as unhatched embryos. |
| | CAA21503 | |
| Y49E10.22 | Z98866 | All F1 worms dead as unhatched embryos. |
| Y39E4B.11 | AL110487 | All F1 worms dead as unhatched embryos. |
| R02F2.7 | U00055 | Arrest at L1/L2 stage: wild-type number of L1/L2-sized F1 worms observed both 3 |
| | | days and 5 days post-injection. |
| C45G9.5 | U21323 | Arrest at L1/L2 stage: fewer than normal L1/L2-sized F1 worms observed both 3 |
| | | days and 5 days post-injection, sugggesting also partial embryonic lethality |
| F23H11.5 | AF003389 | Arrest at L1/L2 stage: wild-type number of L1/L2-sized F1 worms observed both 3 |
| | | days and 5 days post-injection. |
| C32A3.2 | Z48241 | Slowed pace of development, yielding wild-type number of L2/L4-like F1 worms 5 |
| | | days post-injection. |
| K04G7.1 | U21320 | Slowed pace of development, yielding wild-type number of L3-like F1 worms 5 |
| | | days post-injection. |
| K11H3.2 | Z22180 | Slowed pace of development, yielding wild-type number of L3/L4-like F1 worms 5 |
| | | days post-injection. |
| R12B2.5 | U00066 | Slowed pace of development, yielding wild-type number of L3/L4-like F1 worms 5 |
| | | days post-injection. |
| Y39A1A.13 | AL031633 | Adult F1 worms show clear defects falling into several categories: distorted body |
| | | with movements severely uncoordinated in both directions (“unc”), vulval |
| | | deformities, apparent sterility. |
| ZK1236.3 | L13200 | F1 worms die as contorted L3/L4 stage, showing a range of morphological defects |
| | | including vulval deformities, irregular girth, and aberrant internal structures. |
| F23F12.2 | U12965 | Sterility: Injected F0 worms have very few or no embryos |

TABLE 2
Primer pair sequences for generating dsRNAs used for RNAi experiments

| | | dsRNA1 | dsRNA1 | | dsRNA2 | dsRNA2 |
|---|---|---|---|---|---|---|
| Gene Name | dsRNA1 | Upstream primer | Downstream primer | dsRNA2 | Upstream primer | Downstream primer |
| D2045.1 | 320B5 | TCCACAACAAGGACCACCT | GTGACTAGCGTTGCTGCTC | 340G4 | GCAGAAGCAGATGCTGGAT | TGACTGGAACCATTTGTGG |
| C39B5.2 | 332d8 | TGTCAGAGAACTGATGCCG | GTGAAGCGTGAGAAGGTGC | | | |
| C29E4.2 | 307f10 | TGTCAAAATCAATTGCCAC | AAATACCTGGCAGATTCTC | 341c10 | GGCACTCGCCGGATACCTC | AGATTCTCGTCTCCCGTGT |
| H04J21.3 | 338a3 | TTGCAGACTGGAAGCACAC | GCCTAGAAATCCCGAAAAG | 241b7 | CCTTCCACACGTTGAATCT | AGTCAACGCATCCAGTGTG |
| C38C10.4 | 335c2 | GACTGACAGCCTGAAAACA | AACGAGGAACCCGTAGACA | 340f4 | CTCTCTTCTACGCCACGAA | GGACGTCATTGTCACATCA |
| F22B7.13 | 303e1 | CGGTTGTTTTATTGAAGAT | CGAGCTGGAAAAATATAAA | | | |
| C23G10.9 | 330h5 | GAATCTGCCTTTTCTTTGG | GTTTGAACTGCTTTGCCTG | 341d8 | AAGACTCTTATGAAGACCA | GCCCTAGTTTATTTTGACC |
| F54C4.3 | 327c11 | CTCGATTTACCGAATCCAA | CGTCTTCGCTTTCGAATTT | 340d11 | TGAGCAGGACGATGTGGAC | CTATCGTCATTTTCCTCGT |
| K12H4.5 | 329h11 | TTTGGCTCTTTGAAGGCAG | CTCGAATCAGCCTGAAACT | 341f7 | GATCAAGTACCACGTGTCG | AAGAAGATACGGTGCTGGA |
| R13F6.1 | 310h4 | AAATCAAAGCCTTTCCACG | AAATCCACGGCATTTTGTT | | | |
| W07B3.2 | 328e1 | CGAGGAGAGAGCGTCCATA | AGAGAGAAGAGAATGCGGG | 342a1 | CGAGGAGAGAGCGTCCATA | CGAGAAGCGCGTCTACAAT |
| Y66A7A.8 | 323h9 | CCGGGATGTACTTGCACTC | TTACGGGCAGAACTTCCAA | 341b10 | GTTTCTCTGCGTCACTCAT | TTACGGGCAGAACTTCCAA |
| Y49E10.22 | 334h8 | AGCTTGTCGAATCCACGTC | ATTTACGGTGGTGCTCGAT | | | |
| Y39E4B.11 | 338e3 | TCCTCTGTGATACCCCCTG | GAGACGTAAGCGAACGAAG | | | |
| R02F2.7 | 303a11 | AAATACGATTCATTCCTCT | GAAAAACCAATTGCTCTCG | | | |
| C45G9.5 | 308a7 | TATTGATTGAGGTCTCGCA | TAAAATCCGATAATTCGCA | 341c4 | AGCATAAAAATGACCCAAG | TCCTGGTTCAGCTTGCTTC |
| F23H11.5 | 329g11 | ACAATCGACGGAAGTGACA | GACATTTCGGCATTTTTGC | 341g4 | TGAACCGTACCGCTTTCAA | TTTGCGATTGCGATAGCTC |
| C32A3.2 | 325d11 | TGAAACAAATCGCTGCAAA | ACATAGCGATAAGGCGTGG | | | |
| K04G7.1 | 312g8 | CAATTGTTCGTTCTTGTTG | GGTGATGATTTTCCATGAC | 341h10 | CGTTCCGTCGACAACCATA | CTTCATCATTGGTGGATGG |
| K11H3.2 | 326f1 | ATTATCGCTGCTCCAGCTC | GAAATCGCGTCTGAAGTTG | | | |
| R12B2.5 | 304a1 | GTTCCTTTCTTGCTGTTTC | TGAGGACGATCGCAAAAGT | | | |
| Y39A1A.13 | 326e6 | ACCAAGCAGTTTTCCACCT | CGAAAAGCATCAGGAATTG | | | |
| ZK1236.3 | 302e12 | AAGCGAAAATTATGAAGCA | AAAACCTACCTGTTGTGGT | | | |
| F23F12.2 | 309d3 | TTCTCGGTGCTCTTCCATC | GGTAAATGATGAGGACGCT | | | |

1-27. (canceled)

28. A probe comprising a nucleic acid sequence as defined in claim 1 containing a detectable label.

29. The probe of claim 28 being a polynucleotide or an oligonucleotide comprising at least 15 nucleotides.

30. A recombinant vector or nucleic acid construct having incorporated therein the isolated nucleic acid molecule of claim 1 or a fragment thereof.

31. The vector of claim 30 which is an expression vector.

32. A host cell which has been genetically engineered to incorporate therein the isolated nucleic acid molecule of claim 1.

33. A host cell which has been genetically engineered to incorporate therein the recombinant vector or nucleic acid construct of claim 30.

34. The host cell of claim 33 having incorporated therein as an recombinant vector an expression vector.

35. An assay kit comprising the isolated nucleic acid molecule of claim 1 or a fragment thereof in a suitable container.

36. A method for producing a polypeptide which is required for at least one function of nematode worms selected from the group consisting of viability, growth and reproduction, in a host cell comprising the steps
a) transferring the expression vector of claim 31 into a suitable host cell, and
b) cultivating the host cells of step a) under conditions which will permit the expression of said polypeptide or fragment thereof and,
c) optionally, secretion of the expressed polypeptide into the culture medium.

37. The method as claimed in claim 36 wherein the nematode worm is selected from the group consisting of C. elegans and nematode worms parasitic to humans, livestock or plants.

38. A method of producing a polypeptide required for at least one function of nematodes selected from the group consisting of viability, growth and reproduction comprising the step of expressing the isolated nucleic acid molecule or fragment thereof as defined in claim 1.

39. A screening method for drugs that inhibit, stimulate or effect at least a worm function selected from the group consisting of growth, viability and reproduction, comprising the step of contacting said drugs with a nucleic acid molecule as defined in claim 1.

40. A method of treating a human being or animal with disease associated with the infection or presence of nematode worms, comprising the step of administering a medicament comprising a nucleic acid molecule as defined in claim 1.

41. The method of claim 40 wherein the nematode worm is a nematode selected from the group consisting of Wuchereria bancrofti, Brugia malayi, Loa ba, or Onchocerca volvulus.

42. The method of claim 40 wherein the disease is selected from the group consisting of calabar swellings, lymphatic filariasis (elephantiasis) and onchocercoma.

43. A method of diagnosing a human or animal disease associated with the infection or presence of nematode worms comprising the step of hybridizing a nucleic acid molecule as defined in claim 1 with endogenous nucleic acid molecules derived from the human being or animal.

44. The method of claim 43 wherein the disease is selected from the group consisting of calabax swellings, lymphatic filariasis (elephantiasis) and oncho cercoma.

45. A method of treating or diagnosing a plant disease associated with the infection or presence of nematode worms comprising the step of using a nucleic acid molecule as defined in claim 1 or the polypeptides derived therefrom.

46. The method of claim 45 wherein the nematode worm is a nematode selected from the group consisting of Heterodera, including H. glycines, H. avense, H. schachtii, H. trifolii, H. gottingiana, H. cajani, H. zeae; Globodera, including G. rostochiensis, G. pallida, G. tabacum; Meloidogyne, including M. arenaria, M. incognita, M. javanica, M. hapla, M. chitwoodi; Ditylenchus, including D. destructor, D. dipsaci, D. angustus; Anguina, including A. tritici, A. agrostis, Aformna/Anguina wevelli; Pratylenchus, including P. penetrans, P. brachyurus, P. coffeae, P. zeae, P. goodeyi, P. thornei, P. vulnus; Radopholus, including R. similis; Hirschmanniella, including H. oryzae, H. mucronata, H. spinicauda; Hoplolaimus, including H. columbus, H. seinhorsti, H. indicus; Rotylenchulus, including R. reniformis; Tylenchulus, including T. semipenetrans; Helicotylenchus, including H. multicinctus, H. mucronatus, H. dihystera, H. pseudorobustus; Criconemella, including C. xenoplax, C. axestis, C. spharocephalum; Xiphinema, including X. americanum, X. elongatum; Longidorus, including L. africanus; Trichodorus; Paratrichodorus, including P. minor; Aphelenchs, including A. fragariae, A. besseyi, A. ritzemabosi; and Bursaphelenchus xylophilus.

47. A polypeptide required for at least one function of nematodes selected from the group consisting of viability, growth and reproduction, or a fragment thereof comprising an amino acid sequence selected from the group consisting of
a) the amino acid sequences presented in SEQ ID NO. 2, 4, 6, 8, 10, 12, 13, 15, 17, 19, 21, 23, 25, 30, 32, 34, 36, 38, 40, 42, 44, 46, 48 and 50 and fragments thereof,
b) amino acid sequences which exhibit a sequence identity with the sequences of a) of at least 25% over 100 residues and/or which are detectable in a computer aided search using the blast sequence analysis programs with an e-value of at most 10˜30,
c) amino acid sequences encoded by any of the nucleic acid sequences defined as c) to d) in claim 1.

48. An antibody, polyclonal or monoclonal, or a fragment thereof, capable of specifically binding with the polypeptide of claim 47 or with an immunogenic part thereof.

49. A screening method for interacting drugs that inhibit, stimulate or effect at least one function of nematodes selected from the group consisting of viability, growth and reproduction comprising the step of contacting said drugs with polypeptides as claimed in claim 47.

50. The screening method of claim 49 comprising the steps
(1). recombinant expression of said polypeptide in a host cell
(2). isolation and optionally purification of the recombinantly expressed polypeptide of step 1
(3). optionally labelling of the drugs that are tested to interact with said polypeptide and/or labelling of the recombinantly expressed polypeptide
(4). immobilization of the recombinantly expressed polypeptide to a solid phase
(5). binding of a potential interaction partner or a variety thereof to the polypeptide
(6). optionally one or more washing steps
(7). detection and/or quantification of the interaction, in particular by monitoring the amount of label remaining associated with the solid phase over background levels.

51. A method of treating a human being or animals with disease associated with the infection with or the presence of nematode worms, comprising the step of administering a medicament comprising the polypeptides of claim 47.

52. The method of claim 51 wherein the nematode worm is a nematode selected from the group consisting of Wuchereria bancrofti, Brugia malayi, Loa ba and Onchocerca volvulus.

53. The method of claim 51 wherein the disease is selected from the group consisting of calabar swellings, lymphatic filariasis (elephantiasis) and onchocercoma.

54. A method of treating or diagnosing a plant disease associated with the infection with or the presence of nematode worms, comprising the step of using the polypeptide of claim 47.

55. The method of claim 54 wherein the nematode worm is selected from the group consisting of Heterodera, including H. glycines, H. avenae, H. schachtii, H. trifolii, H. gottingiana, H. cajani, H. zeae; Globodera, including G. rostochiensis, G. pallida, G. tabacum; Meloidogyne, including M. arenaria, M. incognita, M. javanica, M. hapla, M. chitwoodi; Ditylenchus, including D. destructor, D. dipsaci, D. angustus; Anguina, including A. tritici, A. agrostis, AfrmnalAnguina wevelli; Pratylenchus, including P. penetrans, P. brachyurus, P. coffeae, P. zeae, P. goodeyi, P. thomei, P. vulnus; Radopholus, including R. similis; Hirschmanniella, including H. oryzae, H. mucronata, H. spinicauda; Hoplolaimus, including H. columbus, H. seinhorsti, H. indicus; Rotylenchulus, including R. reniformis; Tylenchulus, including T. semipenetrans; Helicotylenchus, including H. multicinctus, H. mucronatus, H. dihystera, H. pseudorobustus; Criconemella, including C. xenoplax, C. axestis, C. spharocephalum; Xiphinema, including X. americanum, X. elongatum; Longidorus, including L. africanus; Trichodorus; Paratrichodorus, including P. minor; Aphelenchs, including A. fragariae, A. besseyi, A. ritzemabosi; and Bursaphelenchus xylophilus.

56. A method for evaluating drug binding and efficacy of the polypeptides of claim 46 comprising the step of developing computational models, structural models or other models with the polypeptide sequences as defined in claim 47.
